SEQUENCE LISTING (6 sequences)

SEQ ID NO: 1 - protein, 1880 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVELRGSGLQPDLIEGRKGAQIVKRASLKRGKQ

SEQ ID NO: 2 - DNA, 8300 bp, Homo sapiens
  source 1..8300: /mol_type=“unassigned DNA” /organism=“Homo sapiens”
agaggctgcggtgagtccgccagccccagctgctcctcctcaagcccccaaggcccttcggcgggcccctgctgaaagaccggcatgccctattctgtgggcttccgcgaagccgatgctgctaccagctttctgagagcagcaagatcaggtaacttggacaaagctttggatcacctgcggaatggggtagatattaacacctgtaaccagaatgggttgaatggcttgcatctggcttctaaggaaggccatgtgaaaatggtggttgaacttctgcacaaagaaatcattctagaaacgacaaccaagaaggggaacacggccctgcacatcgctgctctagccgggcaggatgaggtggtccgggagcttgtcaactatggagccaacgtcaacgcccagtcacagaaaggttttacacccctgtacatggcagcacaagagaaccacttggaagtggttaagtttttactggaaaatggagctaaccagaatgtagccacagaagacggcttcacgcctctggcggtagccctgcagcagggccatgagaacgtcgtcgcgcacctcatcaactacggcaccaaggggaaggtgcgcctcccggccctgcacatcgcggcccgcaacgacgacacgcgcacggctgcggtgctgctgcagaacgaccccaacccggacgtgctttccaagacgggattcacgcccctgcacattgcggctcactacgagaacctcaacgtggcccagttgctcctcaacagaggagccagcgtcaatttcacaccacagaacggcatcacgccactgcacatcgcctcccgcaggggcaacgtgatcatggtgcggctgctgctggatcggggagcccagatagaaaccaagaccaaggacgaattgacacctctccactgtgcagctcgaaatgggcacgtgcgaatctcagagatcctgctggaccacggggcaccaatccaagccaaaaccaagaacggcctgtccccaattcacatggcggctcagggagaccacctcgactgtgtccggctcctgttgcaatacgacgcagagatagacgacatcaccctggaccacctgaccccactccacgtggctgcccactgtggacaccacagggtggctaaggtccttctggataaaggggccaaacccaactccagagccctgaatggctttacccccttacacatcgcctgcaaaaagaaccacgtccgtgtcatggagctgctgctgaagacgggagcctcgatcgacgcggtcaccgagtctggcctgacacctctccacgtggcctccttcatggggcaccttcccatcgtgaagaacctcctgcagcggggggcgtcgcccaacgtctccaacgtgaaagtggagaccccgctacacatggcagccagagccgggcacacggaagtggccaaatatttactccagaacaaagccaaagtcaatgccaaggccaaggatgaccagaccccacttcactgtgcagctcgcatcggccacacaaacatggtgaagctcctgctggaaaataacgccaaccccaacctggccaccaccgccgggcacacccccctgcacattgcagcccgtgagggccatgtggaaacagtcctggcccttctggaaaaggaagcatcccaggcctgcatgaccaagaaaggatttacccctctgcacgtggcggccaagtacgggaaggtgcgggtggcagagctgctgctggagcgggacgcacacccgaatgctgccggaaaaaatggcctgacccccctgcacgtggccgtccatcacaacaacctggacatcgtcaagctgctgcttccccggggcggctccccgcacagccctgcctggaatggctacacccctttgcacatcgctgccaagcagaaccaggtggaggtggcccgtagtctgctgcagtatgggggctcagcaaacgccgagtcggtgcaaggtgtgacgccccttcacctggccgcccaggagggccacgcagagatggtggctctgctgctctcgaaacaagccaatggcaacctggggaacaagagcggactcactcccctccatctggtagcacaagaaggccacgttccagtggcagatgtgctgatcaaacacggcgtcatggtggacgccaccacccggatgggctacactcccctccatgtggccagtcactatggaaacatcaagctggtgaagtttctgctgcagcaccaggcagatgtcaatgccaagaccaagctaggatacagccccctgcaccaggcagcccagcagggacacacagacatcgtgactctgcttctgaaaaacggtgcttccccaaacgaggtcagctcggatggaaccacacctctggccatagccaagcgcttgggctacatttctgtcaccgacgtgctcaaggtcgtcacggatgaaaccagtttcgtgttagtcagtgataagcatcgaatgagtttccctgagacagttgatgagatcctggatgtctcggaagatgaaggggaagaactcatcagcttcaaggctgagaggcgggattccagggatgttgatgaagagaaggagctgctggattttgtgccgaagctagaccaagtggtggaatctccagccatccccaggattccctgtgccatgcctgagacagtggtgatcaggtcagaagagcaggagcaggcatctaaagagtatgatgaggactccctcatccccagcagcccggccaccgagacctcagacaacatcagcccggtggccagcccggtgcatacagggtttctggtgagcttcatggttgacgcccggggtggttccatgagaggaagtcgccacaacggcctgcgagtggtgatcccgccacggacgtgcgcagcgcccacccgcatcacctgccgcctggtcaagccccagaagctcagcacgccgcccccactggccgaggaggagggcctggccagcaggatcatagcactggggcccacgggggcacagttcctgagccctgtaatcgtggagatcccgcactttgcctcccatggccgtggagaccgcgagctcgtggttctgaggagcgaaaacggctccgtgtggaaggagcacaggagccgctatggagagagctacctggatcagatcctcaacgggatggacgaagagctggggagcctggaggagctagagaagaagagggtgtgccgaatcatcaccaccgacttcccgctgtacttcgtgatcatgtcacggctctgccaggactacgacaccatcggtcccgaagggggctccctgaagagcaagctggtgcccctggtacaggcaacgttcccggagaatgccgtcaccaagagagtgaagctggctctgcaggcccagcctgtcccggatgagcttgtcactaagctcctgggcaaccaggccacattcagccccattgtcaccgtggagccccggcgccggaagttccaccgccccattgggcttcggatcccactacctccttcctggaccgacaacccgagggacagcggggagggagacaccaccagcctgcgcctgctttgcagcgtcattggaggaacagaccaagcccagtgggaagacataacaggaaccaccaaacttgtatatgccaacgagtgcgccaacttcaccaccaatgtctctgccaggttttggctgtcggactgtcctcggactgctgaggctgtgaactttgccaccctgctgtacaaagagctcactgcagtgccctacatggccaaattcgtcatctttgccaagatgaatgacccccgagaggggcgcctgcgctgctactgcatgacagatgataaagtggacaagaccctggagcagcatgagaacttcgtggaggtggcccggagcagggacatagaggtgttggaaggaatgtccctgtttgcagaactctctgggaacctggtgcctgtgaagaaagctgcccagcagcggagcttccacttccagtcatttcgggagaaccgtctggccatgcctgtaaaggtgagggacagcagtcgagagccgggagggtccctgtcgtttctgcgcaaggcgatgaagtacgaggacacccagcacattctctgccacctgaacatcaccatgcccccctgcgccaagggaagtggagccgaagataggagaaggaccccgacgcccctggccctgcgatacagcattctcagtgagtccacaccaggttctctcagtgggacagagcaggcagagatgaagatggctgttatctcagagcacctcggtctcagctgggcagagttggcccgggagctgcagttcagtgtggaagacatcaacaggatccgagtggaaaatcccaactccctgttggagcagagtgtggccttgctgaacctctgggtcatccgtgaaggccaaaacgcaaacatggagaatctgtacacagccctgcagagcattgaccgtggcgagatcgtgaacatgctggagggttccggccgacagagccgcaacttgaagccagacaggcggcacaccgaccgcgactactcgctgtcaccctcccagatgaatggttactcctcactgcaggacgagctgctgtcccctgcctccctgggctgtgcactttcctctccgctacgtgcagaccagtactggaatgaggtggccgtcctagacgccatccccttggcggccacggagcatgacaccatgctggagatgtctgacatgcaggtgtggtctgcgggcctcacgccttctctggtcactgctgaggactcctctctggagtgtagcaaggctgaggactctgatgccacaggtcacgagtggaagttggagggggcactctcagaggaaccgcggggccccgagttgggctctctggaacttgtggaggacgacacagtggattcagatgccacaaatggccttatcgatttgcttgaacaggaggaaggtcagaggtcagaagagaagctgccaggttctaagaggcaggatgacgcgacaggtgcagggcaggactcagagaatgaagtgtctcttgtttcaggccatcagagggggcaagcccgaatcacacattcccccaccgtgagtcaggtgacggagaggagtcaggacagactgcaggactgggatgcagacggctcgattgtctcatacctgcaagatgctgcacaaggttcctggcaagaggaggtcacgcaaggtccacactcattccagggaacaagtaccatgactgaagggctagagcccggtggatctcaggagtacgagaaggtcctggtgtctgtaagtgagcacacgtggacagaacagcccgaggctgagagctcccaggccgacagggaccggaggcagcaaggccaagaagagcaggtgcaggaggccaagaacaccttcacccaagtggtgcaggggaatgagtttcagaatattccaggggagcaggtgacagaggagcaattcacggatgagcagggcaacattgtcaccaagaagatcattcgcaaggtggttcgacagatagacttgtccagcgccgatgccgcccaggagcacgaggaggtggagctgagagggagtggcctacagccggacctgatagagggcaggaagggggcgcagatagtgaagcgggccagcctgaaaagggggaaacagtgaccccgagccgctctccttggagtagcctctcgggaggatcacacctcgacacccaacccctgaaccccacacactctgccatgcacacaggaggagagctggacctgagggccaccgcagcggtgcacacattcctctgggctgacggcatgacctctgtaagggactcctgctagtcccctcttggcatgaatgactgactgtagacgcatgacctccaggcttcaatcctgcctcttgcaatgacagctgatctgtcggaaccaggacacaaaagcagcaagaagcggggagagagagggatagaaaacaagcgcaggagagcctgcgaacgcaaaagtgaatgagggctttttgtggctggggatgggttttggttttggggttttttttttaaattgttttgacttcgtacagggtactttttcccaacctcatctgtcagaaatccatgtgggcttcctggaaagaaaaaaaaaaaaaagaaaactaggcatgaaatcagtttaacaccttaatcttaagccatgtcctcatctgcccaccctccccaacccacatacctccattccacttgtgacaccccctcgactccctggtgacgctcctcccagatcgctctacatgacattcaggacacacacacacacacacacacaccactcgcctccactgaatctacacacagattttactgtgacttctgaagctgtatagactctgctgtggataaattggaattttttatgttgtctctctctctgccaatttcaatacgaatcatcttccaatggaaaatcattaccttgaagagtgcattcggggttccttgtgttagggacttaagaatctgaggcgaggacccccaggcttacctgtaggactcaaagggagccaggaccccatctgaaagggtctcctctctcagttgggggacaggccggtggctgacccaggattgcaccagcatgtccatagagaagaggttttctatgtcttcaagcactatatcatagtccgtgttcaaagtgtaaactgtacagtaatcagccttgtgtatatgaaaaacaataaatactatgcaaaccaatagaaacatttagcagtacgtacagagcctgacagcgcagctcctgaggacttctgcggctgcaggagaaggagctgtggcctgtctttcagtgaaagaggaaggaaaggagtagggctagtgaatgtatctgccaggtcttagaagtcaggaccacccagggtctcccagagtgaacttggtgctctaagtccaggcagcctgcaggctggggaccctgggtgcaggtgccaggctcgaggctgttccctgtcctgcctcagtgcctcccctctgcccagcccctgtggcctcttggagaggcgagccagggggctccccggaccgcatggccggtgagagtagagtcggaggcggagagcctggacgcccagaggccggggccaggctctgggtcaaggcaggaaggagacagacagctggcagttcctcgcctcggtgccatcagggaggagccttaagttccactagagcctccggcccagggaggccagaatcagcacaatcccggccccgccctcgcggcgggcgcccccagggccaggagagagagacagggctggctcctctcgcccctggagccgcggtgtggacggcccggccccgtccgccccgccgctctgcgggagtgtcctcgggacacacttaaaccttccaagtagcacagaagccccctccacgtcagaggccccctggccgcggggcctgtgagcggagaggggccctgcttcccacacaaatccgccccggtgcacccccggccaggcaggccccggggcggcctaggcgccttgtcggccgggggtggggctgcttttgcttcctttagaggggcgggcagggagaggaagctctagctccgggcctgagtttccccgaggctgcgtcggaggagctgcagggcgagcccggacggctgccgtctccgccagaggccaccccggcccggcctccagggcgcagaggcgcccccctccatcctcccccatcccgggggccacggcaggggggccgcacagcgcggctgggaccgattcggtgacggcccaaggaagctgggctcagggctcgcctctcccgcctcgatcctgcaccttcctctcccaggtcgctgctgcagccaacccagtagcccccttagcgcccccgcggaccccgcagctccagactccgctttggcacctctgctccggctggcatggctgcacgctccggctgctagtaggagccgttaggggaggagtttggggtctccacacgatgcctagagaatgctgcagtctgcacattagacgctttttagaagttttgaaattaccttgatttttttaattgttatgaaaatggatcttttcttgactctcccacatgctctgttatgggagagaatcccctaccctactctgatgtatagaccattctcccttcaccagccgaaccaatgtcaaaattaataaagaaatggactaatggcaaaaaaaaa

SEQ ID NO: 3 - protein, 1881 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVTVEGPLEDPSELEVDIDYFMKHSKDHTSTPNP

SEQ ID NO: 4 - DNA, 8240 bp, Homo sapiens
  source 1..8240: /mol_type=“unassigned DNA” /organism=“Homo sapiens”
agaggctgcggtgagtccgccagccccagctgctcctcctcaagcccccaaggcccttcggcgggcccctgctgaaagaccggcatgccctattctgtgggcttccgcgaagccgatgctgctaccagctttctgagagcagcaagatcaggtaacttggacaaagctttggatcacctgcggaatggggtagatattaacacctgtaaccagaatgggttgaatggcttgcatctggcttctaaggaaggccatgtgaaaatggtggttgaacttctgcacaaagaaatcattctagaaacgacaaccaagaaggggaacacggccctgcacatcgctgctctagccgggcaggatgaggtggtccgggagcttgtcaactatggagccaacgtcaacgcccagtcacagaaaggttttacacccctgtacatggcagcacaagagaaccacttggaagtggttaagtttttactggaaaatggagctaaccagaatgtagccacagaagacggcttcacgcctctggcggtagccctgcagcagggccatgagaacgtcgtcgcgcacctcatcaactacggcaccaaggggaaggtgcgcctcccggccctgcacatcgcggcccgcaacgacgacacgcgcacggctgcggtgctgctgcagaacgaccccaacccggacgtgctttccaagacgggattcacgcccctgcacattgcggctcactacgagaacctcaacgtggcccagttgctcctcaacagaggagccagcgtcaatttcacaccacagaacggcatcacgccactgcacatcgcctcccgcaggggcaacgtgatcatggtgcggctgctgctggatcggggagcccagatagaaaccaagaccaaggacgaattgacacctctccactgtgcagctcgaaatgggcacgtgcgaatctcagagatcctgctggaccacggggcaccaatccaagccaaaaccaagaacggcctgtccccaattcacatggcggctcagggagaccacctcgactgtgtccggctcctgttgcaatacgacgcagagatagacgacatcaccctggaccacctgaccccactccacgtggctgcccactgtggacaccacagggtggctaaggtccttctggataaaggggccaaacccaactccagagccctgaatggctttacccccttacacatcgcctgcaaaaagaaccacgtccgtgtcatggagctgctgctgaagacgggagcctcgatcgacgcggtcaccgagtctggcctgacacctctccacgtggcctccttcatggggcaccttcccatcgtgaagaacctcctgcagcggggggcgtcgcccaacgtctccaacgtgaaagtggagaccccgctacacatggcagccagagccgggcacacggaagtggccaaatatttactccagaacaaagccaaagtcaatgccaaggccaaggatgaccagaccccacttcactgtgcagctcgcatcggccacacaaacatggtgaagctcctgctggaaaataacgccaaccccaacctggccaccaccgccgggcacacccccctgcacattgcagcccgtgagggccatgtggaaacagtcctggcccttctggaaaaggaagcatcccaggcctgcatgaccaagaaaggatttacccctctgcacgtggcggccaagtacgggaaggtgcgggtggcagagctgctgctggagcgggacgcacacccgaatgctgccggaaaaaatggcctgacccccctgcacgtggccgtccatcacaacaacctggacatcgtcaagctgctgcttccccggggcggctccccgcacagccctgcctggaatggctacacccctttgcacatcgctgccaagcagaaccaggtggaggtggcccgtagtctgctgcagtatgggggctcagcaaacgccgagtcggtgcaaggtgtgacgccccttcacctggccgcccaggagggccacgcagagatggtggctctgctgctctcgaaacaagccaatggcaacctggggaacaagagcggactcactcccctccatctggtagcacaagaaggccacgttccagtggcagatgtgctgatcaaacacggcgtcatggtggacgccaccacccggatgggctacactcccctccatgtggccagtcactatggaaacatcaagctggtgaagtttctgctgcagcaccaggcagatgtcaatgccaagaccaagctaggatacagccccctgcaccaggcagcccagcagggacacacagacatcgtgactctgcttctgaaaaacggtgcttccccaaacgaggtcagctcggatggaaccacacctctggccatagccaagcgcttgggctacatttctgtcaccgacgtgctcaaggtcgtcacggatgaaaccagtttcgtgttagtcagtgataagcatcgaatgagtttccctgagacagttgatgagatcctggatgtctcggaagatgaaggggaagaactcatcagcttcaaggctgagaggcgggattccagggatgttgatgaagagaaggagctgctggattttgtgccgaagctagaccaagtggtggaatctccagccatccccaggattccctgtgccatgcctgagacagtggtgatcaggtcagaagagcaggagcaggcatctaaagagtatgatgaggactccctcatccccagcagcccggccaccgagacctcagacaacatcagcccggtggccagcccggtgcatacagggtttctggtgagcttcatggttgacgcccggggtggttccatgagaggaagtcgccacaacggcctgcgagtggtgatcccgccacggacgtgcgcagcgcccacccgcatcacctgccgcctggtcaagccccagaagctcagcacgccgcccccactggccgaggaggagggcctggccagcaggatcatagcactggggcccacgggggcacagttcctgagccctgtaatcgtggagatcccgcactttgcctcccatggccgtggagaccgcgagctcgtggttctgaggagcgaaaacggctccgtgtggaaggagcacaggagccgctatggagagagctacctggatcagatcctcaacgggatggacgaagagctggggagcctggaggagctagagaagaagagggtgtgccgaatcatcaccaccgacttcccgctgtacttcgtgatcatgtcacggctctgccaggactacgacaccatcggtcccgaagggggctccctgaagagcaagctggtgcccctggtacaggcaacgttcccggagaatgccgtcaccaagagagtgaagctggctctgcaggcccagcctgtcccggatgagcttgtcactaagctcctgggcaaccaggccacattcagccccattgtcaccgtggagccccggcgccggaagttccaccgccccattgggcttcggatcccactacctccttcctggaccgacaacccgagggacagcggggagggagacaccaccagcctgcgcctgctttgcagcgtcattggaggaacagaccaagcccagtgggaagacataacaggaaccaccaaacttgtatatgccaacgagtgcgccaacttcaccaccaatgtctctgccaggttttggctgtcggactgtcctcggactgctgaggctgtgaactttgccaccctgctgtacaaagagctcactgcagtgccctacatggccaaattcgtcatctttgccaagatgaatgacccccgagaggggcgcctgcgctgctactgcatgacagatgataaagtggacaagaccctggagcagcatgagaacttcgtggaggtggcccggagcagggacatagaggtgttggaaggaatgtccctgtttgcagaactctctgggaacctggtgcctgtgaagaaagctgcccagcagcggagcttccacttccagtcatttcgggagaaccgtctggccatgcctgtaaaggtgagggacagcagtcgagagccgggagggtccctgtcgtttctgcgcaaggcgatgaagtacgaggacacccagcacattctctgccacctgaacatcaccatgcccccctgcgccaagggaagtggagccgaagataggagaaggaccccgacgcccctggccctgcgatacagcattctcagtgagtccacaccaggttctctcagtgggacagagcaggcagagatgaagatggctgttatctcagagcacctcggtctcagctgggcagagttggcccgggagctgcagttcagtgtggaagacatcaacaggatccgagtggaaaatcccaactccctgttggagcagagtgtggccttgctgaacctctgggtcatccgtgaaggccaaaacgcaaacatggagaatctgtacacagccctgcagagcattgaccgtggcgagatcgtgaacatgctggagggttccggccgacagagccgcaacttgaagccagacaggcggcacaccgaccgcgactactcgctgtcaccctcccagatgaatggttactcctcactgcaggacgagctgctgtcccctgcctccctgggctgtgcactttcctctccgctacgtgcagaccagtactggaatgaggtggccgtcctagacgccatccccttggcggccacggagcatgacaccatgctggagatgtctgacatgcaggtgtggtctgcgggcctcacgccttctctggtcactgctgaggactcctctctggagtgtagcaaggctgaggactctgatgccacaggtcacgagtggaagttggagggggcactctcagaggaaccgcggggccccgagttgggctctctggaacttgtggaggacgacacagtggattcagatgccacaaatggccttatcgatttgcttgaacaggaggaaggtcagaggtcagaagagaagctgccaggttctaagaggcaggatgacgcgacaggtgcagggcaggactcagagaatgaagtgtctcttgtttcaggccatcagagggggcaagcccgaatcacacattcccccaccgtgagtcaggtgacggagaggagtcaggacagactgcaggactgggatgcagacggctcgattgtctcatacctgcaagatgctgcacaaggttcctggcaagaggaggtcacgcaaggtccacactcattccagggaacaagtaccatgactgaagggctagagcccggtggatctcaggagtacgagaaggtcctggtgtctgtaagtgagcacacgtggacagaacagcccgaggctgagagctcccaggccgacagggaccggaggcagcaaggccaagaagagcaggtgcaggaggccaagaacaccttcacccaagtggtgcaggggaatgagtttcagaatattccaggggagcaggtgacagaggagcaattcacggatgagcagggcaacattgtcaccaagaagatcattcgcaaggtggttcgacagatagacttgtccagcgccgatgccgcccaggagcacgaggaggtgactgtagaggggcccctggaggatcccagtgagctggaggtcgatattgattactttatgaaacactccaaggatcacacctcgacacccaacccctgaaccccacacactctgccatgcacacaggaggagagctggacctgagggccaccgcagcggtgcacacattcctctgggctgacggcatgacctctgtaagggactcctgctagtcccctcttggcatgaatgactgactgtagacgcatgacctccaggcttcaatcctgcctcttgcaatgacagctgatctgtcggaaccaggacacaaaagcagcaagaagcggggagagagagggatagaaaacaagcgcaggagagcctgcgaacgcaaaagtgaatgagggctttttgtggctggggatgggttttggttttggggttttttttttaaattgttttgacttcgtacagggtactttttcccaacctcatctgtcagaaatccatgtgggcttcctggaaagaaaaaaaaaaaaaagaaaactaggcatgaaatcagtttaacaccttaatcttaagccatgtcctcatctgcccaccctccccaacccacatacctccattccacttgtgacaccccctcgactccctggtgacgctcctcccagatcgctctacatgacattcaggacacacacacacacacacacacaccactcgcctccactgaatctacacacagattttactgtgacttctgaagctgtatagactctgctgtggataaattggaattttttatgttgtctctctctctgccaatttcaatacgaatcatcttccaatggaaaatcattaccttgaagagtgcattcggggttccttgtgttagggacttaagaatctgaggcgaggacccccaggcttacctgtaggactcaaagggagccaggaccccatctgaaagggtctcctctctcagttgggggacaggccggtggctgacccaggattgcaccagcatgtccatagagaagaggttttctatgtcttcaagcactatatcatagtccgtgttcaaagtgtaaactgtacagtaatcagccttgtgtatatgaaaaacaataaatactatgcaaaccaatagaaacatttagcagtacgtacagagcctgacagcgcagctcctgaggacttctgcggctgcaggagaaggagctgtggcctgtctttcagtgaaagaggaaggaaaggagtagggctagtgaatgtatctgccaggtcttagaagtcaggaccacccagggtctcccagagtgaacttggtgctctaagtccaggcagcctgcaggctggggaccctgggtgcaggtgccaggctcgaggctgttccctgtcctgcctcagtgcctcccctctgcccagcccctgtggcctcttggagaggcgagccagggggctccccggaccgcatggccggtgagagtagagtcggaggcggagagcctggacgcccagaggccggggccaggctctgggtcaaggcaggaaggagacagacagctggcagttcctcgcctcggtgccatcagggaggagccttaagttccactagagcctccggcccagggaggccagaatcagcacaatcccggccccgccctcgcggcgggcgcccccagggccaggagagagagacagggctggctcctctcgcccctggagccgcggtgtggacggcccggccccgtccgccccgccgctctgcgggagtgtcctcgggacacacttaaaccttccaagtagcacagaagccccctccacgtcagaggccccctggccgcggggcctgtgagcggagaggggccctgcttcccacacaaatccgccccggtgcacccccggccaggcaggccccggggcggcctaggcgccttgtcggccgggggtggggctgcttttgcttcctttagaggggcgggcagggagaggaagctctagctccgggcctgagtttccccgaggctgcgtcggaggagctgcagggcgagcccggacggctgccgtctccgccagaggccaccccggcccggcctccagggcgcagaggcgcccccctccatcctcccccatcccgggggccacggcaggggggccgcacagcgcggctgggaccgattcggtgacggcccaaggaagctgggctcagggctcgcctctcccgcctcgatcctgcaccttcctctcccaggtcgctgctgcagccaacccagtagcccccttagcgcccccgcggaccccgcagctccagactccgctttggcacctctgctccggctggcatggctgcacgctccggctgctagtaggagccgttaggggaggagtttggggtctccacacgatgcctagagaatgctgcagtctgcacattagacgctttttagaagttttgaaattaccttgatttttttaattgttatgaaaatggatcttttcttgactctcccacatgctctgttatgggagagaatcccctaccctactctgatgtatagaccattctcccttcaccagccgaaccaatgtcaaaattaataaagaaatggactaatggcaaaaaaaaa

SEQ ID NO: 5 - protein, 155 aa, Homo sapiens
MWTFVTQLLVTLVLLSFFLVSCQNVMHIVRGSLCFVLKHIHQELDKELGESEGLSDDEETISTRVVRRRVFLKGNEFQNIPGEQVTEEQFTDEQGNIVTKKIIRKVVRQIDLSSADAAQEHEEVELRGSGLQPDLIEGRKGAQIVKRASLKRGKQ

SEQ ID NO: 6 - DNA, 3304 bp, Homo sapiens
  source 1..3304: /mol_type=“unassigned DNA” /organism=“Homo sapiens”
gtcacccgggccactcagggcagagcctggctgctgggcagaagggcacttgggccgggctgcttcagccgagagggccattcggtgtcctgcccacagagagagaaacagtgagagaccaagagagcaaaccagagagatagggagggggaatgcaaggggtccctgggggactcaggagtcccagtggagaatgagaggcaggccccccgtggtggccggggccctggctgccagaggaccgggctggtgaggaggcgaggatgtggactttcgtcacccagctgttggtcacgctggtgctgctgagcttcttcctggtcagctgtcagaacgtgatgcacattgtcagggggtccctgtgctttgtgctaaagcacatccaccaggagctggacaaggagctgggggagagcgagggcctcagtgacgacgaggagaccatctccaccagggtggtccggcggcgggtcttcctgaaggggaatgagtttcagaatattccaggggagcaggtgacagaggagcaattcacggatgagcagggcaacattgtcaccaagaagatcattcgcaaggtggttcgacagatagacttgtccagcgccgatgccgcccaggagcacgaggaggtggagctgagagggagtggcctacagccggacctgatagagggcaggaagggggcgcagatagtgaagcgggccagcctgaaaagggggaaacagtgaccccgagccgctctccttggagtagcctctcgggaggatcacacctcgacacccaacccctgaaccccacacactctgccatgcacacaggaggagagctggacctgagggccaccgcagcggtgcacacattcctctgggctgacggcatgacctctgtaagggactcctgctagtcccctcttggcatgaatgactgactgtagacgcatgacctccaggcttcaatcctgcctcttgcaatgacagctgatctgtcggaaccaggacacaaaagcagcaagaagcggggagagagagggatagaaaacaagcgcaggagagcctgcgaacgcaaaagtgaatgagggctttttgtggctggggatgggttttggttttggggttttttttttaaattgttttgacttcgtacagggtactttttcccaacctcatctgtcagaaatccatgtgggcttcctggaaagaaaaaaaaaaaaaagaaaactaggcatgaaatcagtttaacaccttaatcttaagccatgtcctcatctgcccaccctccccaacccacatacctccattccacttgtgacaccccctcgactccctggtgacgctcctcccagatcgctctacatgacattcaggacacacacacacacacacacacaccactcgcctccactgaatctacacacagattttactgtgacttctgaagctgtatagactctgctgtggataaattggaattttttatgttgtctctctctctgccaatttcaatacgaatcatcttccaatggaaaatcattaccttgaagagtgcattcggggttccttgtgttagggacttaagaatctgaggcgaggacccccaggcttacctgtaggactcaaagggagccaggaccccatctgaaagggtctcctctctcagttgggggacaggccggtggctgacccaggattgcaccagcatgtccatagagaagaggttttctatgtcttcaagcactatatcatagtccgtgttcaaagtgtaaactgtacagtaatcagccttgtgtatatgaaaaacaataaatactatgcaaaccaatagaaacatttagcagtacgtacagagcctgacagcgcagctcctgaggacttctgcggctgcaggagaaggagctgtggcctgtctttcagtgaaagaggaaggaaaggagtagggctagtgaatgtatctgccaggtcttagaagtcaggaccacccagggtctcccagagtgaacttggtgctctaagtccaggcagcctgcaggctggggaccctgggtgcaggtgccaggctcgaggctgttccctgtcctgcctcagtgcctcccctctgcccagcccctgtggcctcttggagaggcgagccagggggctccccggaccgcatggccggtgagagtagagtcggaggcggagagcctggacgcccagaggccggggccaggctctgggtcaaggcaggaaggagacagacagctggcagttcctcgcctcggtgccatcagggaggagccttaagttccactagagcctccggcccagggaggccagaatcagcacaatcccggccccgccctcgcggcgggcgcccccagggccaggagagagagacagggctggctcctctcgcccctggagccgcggtgtggacggcccggccccgtccgccccgccgctctgcgggagtgtcctcgggacacacttaaaccttccaagtagcacagaagccccctccacgtcagaggccccctggccgcggggcctgtgagcggagaggggccctgcttcccacacaaatccgccccggtgcacccccggccaggcaggccccggggcggcctaggcgccttgtcggccgggggtggggctgcttttgcttcctttagaggggcgggcagggagaggaagctctagctccgggcctgagtttccccgaggctgcgtcggaggagctgcagggcgagcccggacggctgccgtctccgccagaggccaccccggcccggcctccagggcgcagaggcgcccccctccatcctcccccatcccgggggccacggcaggggggccgcacagcgcggctgggaccgattcggtgacggcccaaggaagctgggctcagggctcgcctctcccgcctcgatcctgcaccttcctctcccaggtcgctgctgcagccaacccagtagcccccttagcgcccccgcggaccccgcagctccagactccgctttggcacctctgctccggctggcatggctgcacgctccggctgctagtaggagccgttaggggaggagtttggggtctccacacgatgcctagagaatgctgcagtctgcacattagacgctttttagaagttttgaaattaccttgatttttttaattgttatgaaaatggatcttttcttgactctcccacatgctctgttatgggagagaatcccctaccctactctgatgtatagaccattctcccttcaccagccgaaccaatgtcaaaattaataaagaaatggactaatggcaaaaaaaaa

(19) United States
(12) Patent Application Publication
(10) Pub. No.: US 2021/0189389 A1
(43) Pub. Date: Jun. 24, 2021

(54) METHODS OF MODULATING ANK1

(71) Applicant: SOCIETE DES PRODUITS NESTLE S.A., Vevey (CH)

(72) Inventors: Armand Valesia, Chavannes-pres-Renens (CH); Nele Gheldof, Cugy (CH); Jorg Hager, Houtaud (FR)

(21) Appl. No.: 16/499,411

(22) PCT Filed: Mar. 29, 2018
(86) PCT No.: PCT/EP2018/058101

§ 371 Date: Sep. 30, 2019

(30) Foreign Application Priority Data
Mar. 31, 2017 (EP) 17164140.0

(51) Int. Cl. C12N 15/113

(52) U.S. Cl. CPC C12N 15/113; C12N 2310/11; C12N 2310/141

(57) ABSTRACT

An agent capable of decreasing the activity of ANK1 for use in (i) reducing fat accumulation and/or (ii) preserving or increasing fat free mass.

### FIELD OF THE INVENTION

The present invention relates to agents which are capable of modulating the activity of ANK1 and the use of such agents in therapy, in particular for (i) reducing fat accumulation and/or (ii) preserving or increasing fat free mass in a subject. The invention also relates to methods of identifying such agents.

### BACKGROUND TO THE INVENTION

Obesity is a chronic metabolic disorder that has reached epidemic proportions in many areas of the world. Obesity is the major risk factor for serious co-morbidities such as type 2 diabetes mellitus, cardiovascular disease, dyslipidaemia and certain types of cancer (World Health Organ. Tech. Rep. Ser. (2000) 894: i-xii, 1-253).

Obesity refers to a condition in which an individual weighs more than usual as a result of excessive accumulation of energy from carbohydrate, fat and the like. The additional weight is typically retained in the form of fat under the skin or around the viscera.

Empirical data suggests that a weight loss of at least 10% of the initial weight results in a considerable decrease in the risk of obesity related co-morbidities (World Health Organ. Tech. Rep. Ser. (2000) 894: i-xii, 1-253). However, the capacity to lose weight shows large inter-subject variability.

Obesity is induced when the amount of energy intake exceeds the amount of energy consumed. Thus, in order to ameliorate obesity, a method of decreasing the amount of energy intake from fat, carbohydrate and the like or a method of increasing the amount of energy consumption by promoting in vivo metabolism is desired. Accordingly, improvements in dietary habit and exercise are considered to be effective methods for the prevention and amelioration of obesity and obesity-related disorders. For example, it has long been recognised that low calorie diet (LCD) interventions can be very efficient in reducing weight and that this weight loss is generally accompanied by an improvement in the risk of obesity related co-morbidities, in particular type 2 diabetes mellitus (World Health Organ. Tech. Rep. Ser. (2000) 894: i-xii, 1-253).

Although a number of methods are known for promoting weight loss, subjects face the risk of regaining lost weight once a period of weight loss intervention has been completed. Such regression risks reducing or potentially completely reversing any benefits that were associated with the loss of weight.

Accordingly, there remains a significant need not only for improved methods of promoting weight loss, but also for methods for supporting weight maintenance (preventing or reducing the regain of lost weight, and hence supporting maintenance of weight at a level similar to that achieved following weight loss intervention). Such improvements would provide more complete treatments for obesity, thus decreasing the risk of obesity-related disorders.

Obesity is associated with a number of physiological changes in the body including differences in the levels of certain gene products, which are either higher or lower in obese subjects than in individuals with a normal body weight (Singla, Bardoloi and Parkash, World J Diabetes (2010)). Moreover, it has been shown that the blood levels for many of these gene products change dramatically during a weight loss intervention (Van Dijk et al. Plos One (2010), Viguerie et al. Plos Genetics (2012)).

However, little is known if these changes in gene expression levels are causally associated with obesity and weight loss or if they are just a reflection of the obese status and the weight loss intervention.

One way to explore a possible causality for changes in gene expression levels is to study if the levels of these genes can be altered in knockdown experiments in animal models. Using modern molecular biology techniques these can be carried out and repeated on several animals. When such knock-down leads to non-lethal phenotypes, the effect of the reduced gene expression can be assessed using physiological, morphological or molecular readouts. Drosophila melanogaster, commonly known as the fruitfly, has been proven a good genetic model system to study the function of specific genes in adipose biology (Hong and Park, Exp. Molec. Medicine, 2010; Pospisilik et al. Cell, 2010; Guo et al. Nature 2008) because of several reasons. First, genetic screens are easy as RNAi knockdown fly lines are readily available for most genes. Second, flies develop rapidly and they can be studied in a high throughput fashion. Third, homology between human and fly genes is rather high. Importantly, fundamental human components and regulating mechanisms of lipid storage and utilization are evolutionarily conserved in the fly. In addition, flies can be subjected to dietary interventions including high sucrose diets, starvation and thus can mimic human dietary lifestyles.

To assess the effect of knockdown of specific genes on metabolism and adipose development in fly, the most established and best readout is accumulation of fat as measured by total triglyceride levels. Body weight per se is less reliable as a readout, as this can be influenced by many factors, including body length, but more importantly difference in fat free mass or muscle mass. Hence, an increased body weight in fly can be positive when it is combined with lower fat accumulation. In addition to absolute fat mass, the ratio of fat mass over muscle mass is therefore preferable as a marker of metabolic health compared to absolute body weight.

### SUMMARY OF THE INVENTION

In one aspect the invention provides an agent capable of decreasing the activity of ANK1 for use in (i) reducing fat accumulation and/or (ii) preserving or increasing fat free mass in a subject.

In one aspect the invention provides an agent capable of decreasing the activity of ANK1 for use in reducing fat accumulation.

In one embodiment, fat accumulation is measured by triglyceride levels.

In one aspect the invention provides an agent capable of decreasing the activity of ANK1 for use in preserving fat free mass in a subject.

In one aspect the invention provides an agent capable of decreasing the activity of ANK1 for use in increasing fat free mass in a subject.

In another aspect, the invention provides an agent capable of decreasing the activity of ANK1 for use in supporting weight maintenance and/or treating or preventing obesity.

In another aspect, the invention provides the use of an agent capable of decreasing the activity of ANK1 for supporting weight maintenance.

In another aspect, the invention provides the use of an agent capable of decreasing the activity of ANK1 for treating or preventing obesity.

In another aspect, the invention provides the use of an agent capable of decreasing the activity of ANK1 for increasing the ratio of fat-free mass to fat mass.

In another aspect, the invention provides the use of an agent capable of decreasing triglyceride levels.

In another aspect, the invention provides the use of an agent capable of decreasing the activity of ANK1 for improving dyslipidemia.

In another aspect, the invention provides the use of an agent capable of reducing risk of cardiovascular disease (CVD).

In another aspect, the invention provides a method of reducing fat accumulation in a subject comprising administering an agent of the invention to a subject in need thereof. In another aspect, the invention provides a method of preserving or increasing fat free mass in a subject comprising administering an agent of the invention to a subject in need thereof. In another aspect, the invention provides a method of supporting weight maintenance comprising administering an agent of the invention to a subject in need thereof. In another aspect, the invention provides a method of reducing fat deposition in a subject comprising administering an agent of the invention to a subject in need thereof. In another aspect, the invention provides a method of treating or preventing obesity comprising administering an agent of the invention to a subject in need thereof.

The activity of ANK1 may be decreased in comparison with the activity in the absence of the agent of the invention. The activity of ANK1 may be decreased by, for example, at least 1%, 2%, 3%, 4%, 5%, 6%, 7%, 8%, 9%, 10%, 15%, 20%, 25%, 50%, 75%, or 100%.

The agent may be, for example, a ANK1 antagonist or inhibitor, or the agent may decrease the level of ANK1 in a cell, preferably an adipose cell.

In one embodiment, the agent is administered to a subject during or after a weight loss intervention. In a preferred embodiment, the agent is administered to a subject during a weight loss intervention. The weight loss intervention may be, for example, a diet regimen (e.g. a low-calorie diet) and/or an exercise regimen.

In one embodiment, a low calorie diet means the consumption by the subject of, on average, not more than 900 kcal per day over a period of at least 6 weeks.

In one embodiment, the low calorie diet comprises the consumption of a meal replacement product.

In one embodiment, the agent decreases the level of ANK1 in a subject. In this context, “level” refers to the amount of ANK1 and may be measured, for example, by analysing the amount of protein expressed and/or by analysing the amount of the corresponding mRNA present. Preferably, the agent decreases the expression of ANK1. For example, siRNAs, shRNAs, miRNAs or antisense RNAs may reduce expression of ANK1.

In one embodiment, siRNAs may reduce expression of ANK1.

The level of ANK1 may be decreased in comparison with the level in the absence of the agent of the invention. The level of ANK1 may be decreased by, for example, at least 1%, 2%, 3%, 4%, 5%, 6%, 7%, 8%, 9%, 10%, 15%, 20%, 25%, 50%, 75% or 100%.

In one embodiment, the agent is selected from the agents listed in Table 1.

In another preferred embodiment, the agent is selected from the group consisting of an siRNA, shRNA, miRNA, antisense RNA, polynucleotide, polypeptide or small molecule. The polypeptide may be, for example, an antibody. Thus, the agent of the invention may be in the form of a polynucleotide encoding an siRNA, shRNA, miRNA or antisense RNA that targets ANK1, or a polypeptide (e.g. an antibody). The polynucleotide may be in the form of a vector, such as a viral vector.

The agent of the invention may be an agent identified by a method of the invention.

In another aspect, the invention provides a method of identifying an agent capable of (i) reducing fat accumulation and/or (ii) preserving or increasing fat free mass in a subject comprising the steps:
(a) contacting a preparation comprising a ANK1 polypeptide or polynucleotide with a candidate agent; and
(b) detecting whether the candidate agent affects the activity of the ANK1 polypeptide or polynucleotide.

The effect on activity of the ANK1 polypeptide or polynucleotide may be analysed by comparing the activities of the ANK1 polypeptide or polynucleotide in the presence and absence (i.e. a control experiment) of the candidate agent.

In another aspect, the invention provides a method of identifying an agent that decreases the activity of ANK1 comprising the steps:
(a) contacting a preparation comprising a ANK1 polypeptide or polynucleotide with a candidate agent; and
(b) detecting whether the candidate agent affects the activity of the ANK1 polypeptide or polynucleotide.

The methods of the invention may be methods for identifying an agent capable of suppressing the appetite of a subject, increasing or prolonging satiety, reducing food intake by a subject and/or reducing fat deposition in a subject.

In one embodiment, the preparation comprising the ANK1 polypeptide or polynucleotide comprises a cell comprising the ANK1 polypeptide or polynucleotide.

In one embodiment, the cell is a muscle cell. In one embodiment, the cell is a brain cell. In a preferred embodiment, the cell is an adipocyte.

In one embodiment, the method is for identifying an agent that decreases the expression of ANK1.

In one embodiment, the candidate agent is a natural product, preferably a compound naturally occurring in plants.

In another aspect, the invention provides the use of ANK1, or a polynucleotide encoding the same, in a method of identifying an agent that supports weight maintenance, suppresses the appetite of a subject, increases or prolongs satiety, reduces food intake by a subject, reduces fat deposition in a subject, and/or treats or prevents obesity.

In another aspect, the invention provides the use of an agent capable of decreasing the activity of ANK1 for manufacturing a medicament for use in reducing fat accumulation, preserving or increasing fat free mass in a subject, supporting weight maintenance, suppressing the appetite of a subject, increasing or prolonging satiety, reducing food intake by a subject, reducing fat deposition in a subject, and/or treating or preventing obesity.

In another aspect, the invention provides a method of identifying an agent that decreases the expression of ANK1 comprising the steps:
(a) contacting a cell, preferably a cell expressing the ANK1, with a candidate agent; and
(b) detecting whether the candidate agent decreases the expression of the ANK1.

### DESCRIPTION OF THE DRAWINGS

FIG. 1

A Manhattan plot identifying the region of the ANK1 gene on chromosome 8. Variant position is indicated on the X-axis, statistical significance (−log 10 p-value) is indicated on the Y axis. Each point corresponds to a variant. Gene positions are indicated in the lower panel. The peak lines in the upper panel (secondary Y-axis on the right) indicate recombination rates (in centimorgans per megabase). The top associated variant is indicated with a diamond shape and its chromosome coordinate.

FIG. 2

Whole body RNAi knockdown of Ank increase body weight and reduce triglycerides in Drosophila. (A) Ank mRNA expression reduced by 75% in Ank whole body RNAi flies. (B) Body weight increased in Ank RNAi flies. (C) TAG decreased in Ank RNAi flies. (D) Fly body length has no change in Ank RNAi flies. Data are represented as means±SEM. The light gray bars show data for the wild-type fly (Actin-Gal4/+) and the dark gray bars show data for the RNAi knockdown fly (Actin-Gal4>UAS-Ank^{IR}) Unpaired t test. *, p<0.05;****, p<0.0001, n.s., not significant.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

### ANK1

Ankyrins are a family of proteins that link the integral membrane proteins to the underlying spectrin-actin cytoskeleton and play key roles in activities such as cell motility, activation, proliferation, contact and the maintenance of specialized membrane domains. Multiple isoforms of ankyrin with different affinities for various target proteins are expressed in a tissue-specific, developmentally regulated manner. Most ankyrins are typically composed of three structural domains: an amino-terminal domain containing multiple ankyrin repeats; a central region with a highly conserved spectrin binding domain; and a carboxy-terminal regulatory domain which is the least conserved and subject to variation.

Ankyrin 1, the prototype of this family, was first discovered in the erythrocytes, but since has also been found in brain and muscles. Mutations in erythrocytic ankyrin 1 have been associated in approximately half of all patients with hereditary spherocytosis. Complex patterns of alternative splicing in the regulatory domain, giving rise to different isoforms of ankyrin 1 have been described. Truncated muscle-specific isoforms of ankyrin 1 resulting from usage of an alternate promoter have also been identified.

In Human, the ANK1 gene is also known as ANK; SPH1; SPH2.

In one embodiment, the ANK1 is human ANK1.

An example amino acid sequence of the ANK1 is the sequence deposited under NCBI Accession No. NP_000028.3 An example amino acid sequence of the ANK1 is:

(SEQ ID NO: 1)
MPYSVGFREADAATSFLRAARSGNLDKALDHLRNGVDINTCNQNGLNGLH
LASKEGHVKMVVELLHKEIILETTTKKGNTALHIAALAGQDEVVRELVNY
GANVNAQSQKGFTPLYMAAQENHLEVVKFLLENGANQNVATEDGFTPLAV
ALQQGHENVVAHLINYGTKGKVRLPALHIAARNDDTRTAAVLLQNDPNPD
VLSKTGFTPLHIAAHYENLNVAQLLLNRGASVNFTPQNGITPLHIASRRG
NVIMVRLLLDRGAQIETKTKDELTPLHCAARNGHVRISEILLDHGAPIQA
KTKNGLSPIHMAAQGDHLDCVRLLLQYDAEIDDITLDHLTPLHVAAHCGH
HRVAKVLLDKGAKPNSRALNGFTPLHIACKKNHVRVMELLLKTGASIDAV
TESGLTPLHVASFMGHLPIVKNLLQRGASPNVSNVKVETPLHMAARAGHT
EVAKYLLQNKAKVNAKAKDDQTPLHCAARIGHTNMVKLLLENNANPNLAT
TAGHTPLHIAAREGHVETVLALLEKEASQACMTKKGFTPLHVAAKYGKVR
VAELLLERDAHPNAAGKNGLTPLHVAVHHNNLDIVKLLLPRGGSPHSPAW
NGYTPLHIAAKQNQVEVARSLLQYGGSANAESVQGVTPLHLAAQEGHAEM
VALLLSKQANGNLGNKSGLTPLHLVAQEGHVPVADVLIKHGVMVDATTRM
GYTPLHVASHYGNIKLVKFLLQHQADVNAKTKLGYSPLHQAAQQGHTDIV
TLLLKNGASPNEVSSDGTTPLAIAKRLGYISVTDVLKVVTDETSFVLVSD
KHRMSFPETVDEILDVSEDEGEELISFKAERRDSRDVDEEKELLDFVPKL
DQVVESPAIPRIPCAMPETVVIRSEEQEQASKEYDEDSLIPSSPATETSD
NISPVASPVHTGFLVSFMVDARGGSMRGSRHNGLRVVIPPRTCAAPTRIT
CRLVKPQKLSTPPPLAEEEGLASRIIALGPTGAQFLSPVIVEIPHFASHG
RGDRELVVLRSENGSVWKEHRSRYGESYLDQILNGMDEELGSLEELEKKR
VCRIITTDFPLYFVIMSRLCQDYDTIGPEGGSLKSKLVPLVQATFPENAV
TKRVKLALQAQPVPDELVTKLLGNQATFSPIVTVEPRRRKFHRPIGLRIP
LPPSWTDNPRDSGEGDTTSLRLLCSVIGGTDQAQWEDITGTTKLVYANEC
ANFTTNVSARFWLSDCPRTAEAVNFATLLYKELTAVPYMAKFVIFAKMND
PREGRLRCYCMTDDKVDKTLEQHENFVEVARSRDIEVLEGMSLFAELSGN
LVPVKKAAQQRSFHFQSFRENRLAMPVKVRDSSREPGGSLSFLRKAMKYE
DTQHILCHLNITMPPCAKGSGAEDRRRTPTPLALRYSILSESTPGSLSGT
EQAEMKMAVISEHLGLSWAELARELQFSVEDINRIRVENPNSLLEQSVAL
LNLWVIREGQNANMENLYTALQSIDRGEIVNMLEGSGRQSRNLKPDRRHT
DRDYSLSPSQMNGYSSLQDELLSPASLGCALSSPLRADQYWNEVAVLDAI
PLAATEHDTMLEMSDMQVWSAGLTPSLVTAEDSSLECSKAEDSDATGHEW
KLEGALSEEPRGPELGSLELVEDDTVDSDATNGLIDLLEQEEGQRSEEKL
PGSKRQDDATGAGQDSENEVSLVSGHQRGQARITHSPTVSQVTERSQDRL
QDWDADGSIVSYLQDAAQGSWQEEVTQGPHSFQGTSTMTEGLEPGGSQEY
EKVLVSVSEHTWTEQPEAESSQADRDRRQQGQEEQVQEAKNTFTQVVQGN
EFQNIPGEQVTEEQFTDEQGNIVTKKIIRKVVRQIDLSSADAAQEHEEVE
LRGSGLQPDLIEGRKGAQIVKRASLKRGKQ

An example nucleotide sequence encoding the ANK1 is the sequence deposited under NCBI Accession No. NM_000037.3.

An example nucleotide sequence encoding the ANK1 is:

(SEQ ID NO: 2)
AGAGGCTGCGGTGAGTCCGCCAGCCCCAGCTGCTCCTCCTCAAGCCCCCA
AGGCCCTTCGGCGGGCCCCTGCTGAAAGACCGGCATGCCCTATTCTGTGG
GCTTCCGCGAAGCCGATGCTGCTACCAGCTTTCTGAGAGCAGCAAGATCA
GGTAACTTGGACAAAGCTTTGGATCACCTGCGGAATGGGGTAGATATTAA
CACCTGTAACCAGAATGGGTTGAATGGCTTGCATCTGGCTTCTAAGGAAG
GCCATGTGAAAATGGTGGTTGAACTTCTGCACAAAGAAATCATTCTAGAA
ACGACAACCAAGAAGGGGAACACGGCCCTGCACATCGCTGCTCTAGCCGG
GCAGGATGAGGTGGTCCGGGAGCTTGTCAACTATGGAGCCAACGTCAACG
CCCAGTCACAGAAAGGTTTTACACCCCTGTACATGGCAGCACAAGAGAAC
CACTTGGAAGTGGTTAAGTTTTTACTGGAAAATGGAGCTAACCAGAATGT
AGCCACAGAAGACGGCTTCACGCCTCTGGCGGTAGCCCTGCAGCAGGGCC
ATGAGAACGTCGTCGCGCACCTCATCAACTACGGCACCAAGGGGAAGGTG
CGCCTCCCGGCCCTGCACATCGCGGCCCGCAACGACGACACGCGCACGGC
TGCGGTGCTGCTGCAGAACGACCCCAACCCGGACGTGCTTTCCAAGACGG
GATTCACGCCCCTGCACATTGCGGCTCACTACGAGAACCTCAACGTGGCC
CAGTTGCTCCTCAACAGAGGAGCCAGCGTCAATTTCACACCACAGAACGG
CATCACGCCACTGCACATCGCCTCCCGCAGGGGCAACGTGATCATGGTGC
GGCTGCTGCTGGATCGGGGAGCCCAGATAGAAACCAAGACCAAGGACGAA
TTGACACCTCTCCACTGTGCAGCTCGAAATGGGCACGTGCGAATCTCAGA
GATCCTGCTGGACCACGGGGCACCAATCCAAGCCAAAACCAAGAACGGCC
TGTCCCCAATTCACATGGCGGCTCAGGGAGACCACCTCGACTGTGTCCGG
CTCCTGTTGCAATACGACGCAGAGATAGACGACATCACCCTGGACCACCT
GACCCCACTCCACGTGGCTGCCCACTGTGGACACCACAGGGTGGCTAAGG
TCCTTCTGGATAAAGGGGCCAAACCCAACTCCAGAGCCCTGAATGGCTTT
ACCCCCTTACACATCGCCTGCAAAAAGAACCACGTCCGTGTCATGGAGCT
GCTGCTGAAGACGGGAGCCTCGATCGACGCGGTCACCGAGTCTGGCCTGA
CACCTCTCCACGTGGCCTCCTTCATGGGGCACCTTCCCATCGTGAAGAAC
CTCCTGCAGCGGGGGGCGTCGCCCAACGTCTCCAACGTGAAAGTGGAGAC
CCCGCTACACATGGCAGCCAGAGCCGGGCACACGGAAGTGGCCAAATATT
TACTCCAGAACAAAGCCAAAGTCAATGCCAAGGCCAAGGATGACCAGACC
CCACTTCACTGTGCAGCTCGCATCGGCCACACAAACATGGTGAAGCTCCT
GCTGGAAAATAACGCCAACCCCAACCTGGCCACCACCGCCGGGCACACCC
CCCTGCACATTGCAGCCCGTGAGGGCCATGTGGAAACAGTCCTGGCCCTT
CTGGAAAAGGAAGCATCCCAGGCCTGCATGACCAAGAAAGGATTTACCCC
TCTGCACGTGGCGGCCAAGTACGGGAAGGTGCGGGTGGCAGAGCTGCTGC
TGGAGCGGGACGCACACCCGAATGCTGCCGGAAAAAATGGCCTGACCCCC
CTGCACGTGGCCGTCCATCACAACAACCTGGACATCGTCAAGCTGCTGCT
TCCCCGGGGCGGCTCCCCGCACAGCCCTGCCTGGAATGGCTACACCCCTT
TGCACATCGCTGCCAAGCAGAACCAGGTGGAGGTGGCCCGTAGTCTGCTG
CAGTATGGGGGCTCAGCAAACGCCGAGTCGGTGCAAGGTGTGACGCCCCT
TCACCTGGCCGCCCAGGAGGGCCACGCAGAGATGGTGGCTCTGCTGCTCT
CGAAACAAGCCAATGGCAACCTGGGGAACAAGAGCGGACTCACTCCCCTC
CATCTGGTAGCACAAGAAGGCCACGTTCCAGTGGCAGATGTGCTGATCAA
ACACGGCGTCATGGTGGACGCCACCACCCGGATGGGCTACACTCCCCTCC
ATGTGGCCAGTCACTATGGAAACATCAAGCTGGTGAAGTTTCTGCTGCAG
CACCAGGCAGATGTCAATGCCAAGACCAAGCTAGGATACAGCCCCCTGCA
CCAGGCAGCCCAGCAGGGACACACAGACATCGTGACTCTGCTTCTGAAAA
ACGGTGCTTCCCCAAACGAGGTCAGCTCGGATGGAACCACACCTCTGGCC
ATAGCCAAGCGCTTGGGCTACATTTCTGTCACCGACGTGCTCAAGGTCGT
CACGGATGAAACCAGTTTCGTGTTAGTCAGTGATAAGCATCGAATGAGTT
TCCCTGAGACAGTTGATGAGATCCTGGATGTCTCGGAAGATGAAGGGGAA
GAACTCATCAGCTTCAAGGCTGAGAGGCGGGATTCCAGGGATGTTGATGA
AGAGAAGGAGCTGCTGGATTTTGTGCCGAAGCTAGACCAAGTGGTGGAAT
CTCCAGCCATCCCCAGGATTCCCTGTGCCATGCCTGAGACAGTGGTGATC
AGGTCAGAAGAGCAGGAGCAGGCATCTAAAGAGTATGATGAGGACTCCCT
CATCCCCAGCAGCCCGGCCACCGAGACCTCAGACAACATCAGCCCGGTGG
CCAGCCCGGTGCATACAGGGTTTCTGGTGAGCTTCATGGTTGACGCCCGG
GGTGGTTCCATGAGAGGAAGTCGCCACAACGGCCTGCGAGTGGTGATCCC
GCCACGGACGTGCGCAGCGCCCACCCGCATCACCTGCCGCCTGGTCAAGC
CCCAGAAGCTCAGCACGCCGCCCCCACTGGCCGAGGAGGAGGGCCTGGCC
AGCAGGATCATAGCACTGGGGCCCACGGGGGCACAGTTCCTGAGCCCTGT
AATCGTGGAGATCCCGCACTTTGCCTCCCATGGCCGTGGAGACCGCGAGC
TCGTGGTTCTGAGGAGCGAAAACGGCTCCGTGTGGAAGGAGCACAGGAGC
CGCTATGGAGAGAGCTACCTGGATCAGATCCTCAACGGGATGGACGAAGA
GCTGGGGAGCCTGGAGGAGCTAGAGAAGAAGAGGGTGTGCCGAATCATCA
CCACCGACTTCCCGCTGTACTTCGTGATCATGTCACGGCTCTGCCAGGAC
TACGACACCATCGGTCCCGAAGGGGGCTCCCTGAAGAGCAAGCTGGTGCC
CCTGGTACAGGCAACGTTCCCGGAGAATGCCGTCACCAAGAGAGTGAAGC
TGGCTCTGCAGGCCCAGCCTGTCCCGGATGAGCTTGTCACTAAGCTCCTG
GGCAACCAGGCCACATTCAGCCCCATTGTCACCGTGGAGCCCCGGCGCCG
GAAGTTCCACCGCCCCATTGGGCTTCGGATCCCACTACCTCCTTCCTGGA
CCGACAACCCGAGGGACAGCGGGGAGGGAGACACCACCAGCCTGCGCCTG
CTTTGCAGCGTCATTGGAGGAACAGACCAAGCCCAGTGGGAAGACATAAC
AGGAACCACCAAACTTGTATATGCCAACGAGTGCGCCAACTTCACCACCA
ATGTCTCTGCCAGGTTTTGGCTGTCGGACTGTCCTCGGACTGCTGAGGCT
GTGAACTTTGCCACCCTGCTGTACAAAGAGCTCACTGCAGTGCCCTACAT
GGCCAAATTCGTCATCTTTGCCAAGATGAATGACCCCCGAGAGGGGCGCC
TGCGCTGCTACTGCATGACAGATGATAAAGTGGACAAGACCCTGGAGCAG
CATGAGAACTTCGTGGAGGTGGCCCGGAGCAGGGACATAGAGGTGTTGGA
AGGAATGTCCCTGTTTGCAGAACTCTCTGGGAACCTGGTGCCTGTGAAGA
AAGCTGCCCAGCAGCGGAGCTTCCACTTCCAGTCATTTCGGGAGAACCGT
CTGGCCATGCCTGTAAAGGTGAGGGACAGCAGTCGAGAGCCGGGAGGGTC
CCTGTCGTTTCTGCGCAAGGCGATGAAGTACGAGGACACCCAGCACATTC
TCTGCCACCTGAACATCACCATGCCCCCCTGCGCCAAGGGAAGTGGAGCC
GAAGATAGGAGAAGGACCCCGACGCCCCTGGCCCTGCGATACAGCATTCT
CAGTGAGTCCACACCAGGTTCTCTCAGTGGGACAGAGCAGGCAGAGATGA
AGATGGCTGTTATCTCAGAGCACCTCGGTCTCAGCTGGGCAGAGTTGGCC
CGGGAGCTGCAGTTCAGTGTGGAAGACATCAACAGGATCCGAGTGGAAAA
TCCCAACTCCCTGTTGGAGCAGAGTGTGGCCTTGCTGAACCTCTGGGTCA
TCCGTGAAGGCCAAAACGCAAACATGGAGAATCTGTACACAGCCCTGCAG
AGCATTGACCGTGGCGAGATCGTGAACATGCTGGAGGGTTCCGGCCGACA
GAGCCGCAACTTGAAGCCAGACAGGCGGCACACCGACCGCGACTACTCGC
TGTCACCCTCCCAGATGAATGGTTACTCCTCACTGCAGGACGAGCTGCTG
TCCCCTGCCTCCCTGGGCTGTGCACTTTCCTCTCCGCTACGTGCAGACCA
GTACTGGAATGAGGTGGCCGTCCTAGACGCCATCCCCTTGGCGGCCACGG
AGCATGACACCATGCTGGAGATGTCTGACATGCAGGTGTGGTCTGCGGGC
CTCACGCCTTCTCTGGTCACTGCTGAGGACTCCTCTCTGGAGTGTAGCAA
GGCTGAGGACTCTGATGCCACAGGTCACGAGTGGAAGTTGGAGGGGGCAC
TCTCAGAGGAACCGCGGGGCCCCGAGTTGGGCTCTCTGGAACTTGTGGAG
GACGACACAGTGGATTCAGATGCCACAAATGGCCTTATCGATTTGCTTGA
ACAGGAGGAAGGTCAGAGGTCAGAAGAGAAGCTGCCAGGTTCTAAGAGGC
AGGATGACGCGACAGGTGCAGGGCAGGACTCAGAGAATGAAGTGTCTCTT
GTTTCAGGCCATCAGAGGGGGCAAGCCCGAATCACACATTCCCCCACCGT
GAGTCAGGTGACGGAGAGGAGTCAGGACAGACTGCAGGACTGGGATGCAG
ACGGCTCGATTGTCTCATACCTGCAAGATGCTGCACAAGGTTCCTGGCAA
GAGGAGGTCACGCAAGGTCCACACTCATTCCAGGGAACAAGTACCATGAC
TGAAGGGCTAGAGCCCGGTGGATCTCAGGAGTACGAGAAGGTCCTGGTGT
CTGTAAGTGAGCACACGTGGACAGAACAGCCCGAGGCTGAGAGCTCCCAG
GCCGACAGGGACCGGAGGCAGCAAGGCCAAGAAGAGCAGGTGCAGGAGGC
CAAGAACACCTTCACCCAAGTGGTGCAGGGGAATGAGTTTCAGAATATTC
CAGGGGAGCAGGTGACAGAGGAGCAATTCACGGATGAGCAGGGCAACATT
GTCACCAAGAAGATCATTCGCAAGGTGGTTCGACAGATAGACTTGTCCAG
CGCCGATGCCGCCCAGGAGCACGAGGAGGTGGAGCTGAGAGGGAGTGGCC
TACAGCCGGACCTGATAGAGGGCAGGAAGGGGGCGCAGATAGTGAAGCGG
GCCAGCCTGAAAAGGGGGAAACAGTGACCCCGAGCCGCTCTCCTTGGAGT
AGCCTCTCGGGAGGATCACACCTCGACACCCAACCCCTGAACCCCACACA
CTCTGCCATGCACACAGGAGGAGAGCTGGACCTGAGGGCCACCGCAGCGG
TGCACACATTCCTCTGGGCTGACGGCATGACCTCTGTAAGGGACTCCTGC
TAGTCCCCTCTTGGCATGAATGACTGACTGTAGACGCATGACCTCCAGGC
TTCAATCCTGCCTCTTGCAATGACAGCTGATCTGTCGGAACCAGGACACA
AAAGCAGCAAGAAGCGGGGAGAGAGAGGGATAGAAAACAAGCGCAGGAGA
GCCTGCGAACGCAAAAGTGAATGAGGGCTTTTTGTGGCTGGGGATGGGTT
TTGGTTTTGGGGTTTTTTTTTTAAATTGTTTTGACTTCGTACAGGGTACT
TTTTCCCAACCTCATCTGTCAGAAATCCATGTGGGCTTCCTGGAAAGAAA
AAAAAAAAAAAGAAAACTAGGCATGAAATCAGTTTAACACCTTAATCTTA
AGCCATGTCCTCATCTGCCCACCCTCCCCAACCCACATACCTCCATTCCA
CTTGTGACACCCCCTCGACTCCCTGGTGACGCTCCTCCCAGATCGCTCTA
CATGACATTCAGGACACACACACACACACACACACACCACTCGCCTCCAC
TGAATCTACACACAGATTTTACTGTGACTTCTGAAGCTGTATAGACTCTG
CTGTGGATAAATTGGAATTTTTTATGTTGTCTCTCTCTCTGCCAATTTCA
ATACGAATCATCTTCCAATGGAAAATCATTACCTTGAAGAGTGCATTCGG
GGTTCCTTGTGTTAGGGACTTAAGAATCTGAGGCGAGGACCCCCAGGCTT
ACCTGTAGGACTCAAAGGGAGCCAGGACCCCATCTGAAAGGGTCTCCTCT
CTCAGTTGGGGGACAGGCCGGTGGCTGACCCAGGATTGCACCAGCATGTC
CATAGAGAAGAGGTTTTCTATGTCTTCAAGCACTATATCATAGTCCGTGT
TCAAAGTGTAAACTGTACAGTAATCAGCCTTGTGTATATGAAAAACAATA
AATACTATGCAAACCAATAGAAACATTTAGCAGTACGTACAGAGCCTGAC
AGCGCAGCTCCTGAGGACTTCTGCGGCTGCAGGAGAAGGAGCTGTGGCCT
GTCTTTCAGTGAAAGAGGAAGGAAAGGAGTAGGGCTAGTGAATGTATCTG
CCAGGTCTTAGAAGTCAGGACCACCCAGGGTCTCCCAGAGTGAACTTGGT
GCTCTAAGTCCAGGCAGCCTGCAGGCTGGGGACCCTGGGTGCAGGTGCCA
GGCTCGAGGCTGTTCCCTGTCCTGCCTCAGTGCCTCCCCTCTGCCCAGCC
CCTGTGGCCTCTTGGAGAGGCGAGCCAGGGGGCTCCCCGGACCGCATGGC
CGGTGAGAGTAGAGTCGGAGGCGGAGAGCCTGGACGCCCAGAGGCCGGGG
CCAGGCTCTGGGTCAAGGCAGGAAGGAGACAGACAGCTGGCAGTTCCTCG
CCTCGGTGCCATCAGGGAGGAGCCTTAAGTTCCACTAGAGCCTCCGGCCC
AGGGAGGCCAGAATCAGCACAATCCCGGCCCCGCCCTCGCGGCGGGCGCC
CCCAGGGCCAGGAGAGAGAGACAGGGCTGGCTCCTCTCGCCCCTGGAGCC
GCGGTGTGGACGGCCCGGCCCCGTCCGCCCCGCCGCTCTGCGGGAGTGTC
CTCGGGACACACTTAAACCTTCCAAGTAGCACAGAAGCCCCCTCCACGTC
AGAGGCCCCCTGGCCGCGGGGCCTGTGAGCGGAGAGGGGCCCTGCTTCCC
ACACAAATCCGCCCCGGTGCACCCCCGGCCAGGCAGGCCCCGGGGCGGCC
TAGGCGCCTTGTCGGCCGGGGGTGGGGCTGCTTTTGCTTCCTTTAGAGGG
GCGGGCAGGGAGAGGAAGCTCTAGCTCCGGGCCTGAGTTTCCCCGAGGCT
GCGTCGGAGGAGCTGCAGGGCGAGCCCGGACGGCTGCCGTCTCCGCCAGA
GGCCACCCCGGCCCGGCCTCCAGGGCGCAGAGGCGCCCCCCTCCATCCTC
CCCCATCCCGGGGGCCACGGCAGGGGGGCCGCACAGCGCGGCTGGGACCG
ATTCGGTGACGGCCCAAGGAAGCTGGGCTCAGGGCTCGCCTCTCCCGCCT
CGATCCTGCACCTTCCTCTCCCAGGTCGCTGCTGCAGCCAACCCAGTAGC
CCCCTTAGCGCCCCCGCGGACCCCGCAGCTCCAGACTCCGCTTTGGCACC
TCTGCTCCGGCTGGCATGGCTGCACGCTCCGGCTGCTAGTAGGAGCCGTT
AGGGGAGGAGTTTGGGGTCTCCACACGATGCCTAGAGAATGCTGCAGTCT
GCACATTAGACGCTTTTTAGAAGTTTTGAAATTACCTTGATTTTTTTAAT
TGTTATGAAAATGGATCTTTTCTTGACTCTCCCACATGCTCTGTTATGGG
AGAGAATCCCCTACCCTACTCTGATGTATAGACCATTCTCCCTTCACCAG
CCGAACCAATGTCAAAATTAATAAAGAAATGGACTAATGGCAAAAAAAAA

A further example amino acid sequence of the ANK1 is the sequence deposited under NCBI Accession No. NP_065209.2.

A further example amino acid sequence of the ANK1 is:

(SEQ ID NO: 3)
MPYSVGFREADAATSFLRAARSGNLDKALDHLRNGVDINTCNQNGLNGLH
LASKEGHVKMVVELLHKEIILETTTKKGNTALHIAALAGQDEVVRELVNY
GANVNAQSQKGFTPLYMAAQENHLEVVKFLLENGANQNVATEDGFTPLAV
ALQQGHENVVAHLINYGTKGKVRLPALHIAARNDDTRTAAVLLQNDPNPD
VLSKTGFTPLHIAAHYENLNVAQLLLNRGASVNFTPQNGITPLHIASRRG
NVIMVRLLLDRGAQIETKTKDELTPLHCAARNGHVRISEILLDHGAPIQA
KTKNGLSPIHMAAQGDHLDCVRLLLQYDAEIDDITLDHLTPLHVAAHCGH
HRVAKVLLDKGAKPNSRALNGFTPLHIACKKNHVRVMELLLKTGASIDAV
TESGLTPLHVASFMGHLPIVKNLLQRGASPNVSNVKVETPLHMAARAGHT
EVAKYLLQNKAKVNAKAKDDQTPLHCAARIGHTNMVKLLLENNANPNLAT
TAGHTPLHIAAREGHVETVLALLEKEASQACMTKKGFTPLHVAAKYGKVR
VAELLLERDAHPNAAGKNGLTPLHVAVHHNNLDIVKLLLPRGGSPHSPAW
NGYTPLHIAAKQNQVEVARSLLQYGGSANAESVQGVTPLHLAAQEGHAEM
VALLLSKQANGNLGNKSGLTPLHLVAQEGHVPVADVLIKHGVMVDATTRM
GYTPLHVASHYGNIKLVKFLLQHQADVNAKTKLGYSPLHQAAQQGHTDIV
TLLLKNGASPNEVSSDGTTPLAIAKRLGYISVTDVLKVVTDETSFVLVSD
KHRMSFPETVDEILDVSEDEGEELISFKAERRDSRDVDEEKELLDFVPKL
DQVVESPAIPRIPCAMPETVVIRSEEQEQASKEYDEDSLIPSSPATETSD
NISPVASPVHTGFLVSFMVDARGGSMRGSRHNGLRVVIPPRTCAAPTRIT
CRLVKPQKLSTPPPLAEEEGLASRIIALGPTGAQFLSPVIVEIPHFASHG
RGDRELVVLRSENGSVWKEHRSRYGESYLDQILNGMDEELGSLEELEKKR
VCRIITTDFPLYFVIMSRLCQDYDTIGPEGGSLKSKLVPLVQATFPENAV
TKRVKLALQAQPVPDELVTKLLGNQATFSPIVTVEPRRRKFHRPIGLRIP
LPPSWTDNPRDSGEGDTTSLRLLCSVIGGTDQAQWEDITGTTKLVYANEC
ANFTTNVSARFWLSDCPRTAEAVNFATLLYKELTAVPYMAKFVIFAKMND
PREGRLRCYCMTDDKVDKTLEQHENFVEVARSRDIEVLEGMSLFAELSGN
LVPVKKAAQQRSFHFQSFRENRLAMPVKVRDSSREPGGSLSFLRKAMKYE
DTQHILCHLNITMPPCAKGSGAEDRRRTPTPLALRYSILSESTPGSLSGT
EQAEMKMAVISEHLGLSWAELARELQFSVEDINRIRVENPNSLLEQSVAL
LNLWVIREGQNANMENLYTALQSIDRGEIVNMLEGSGRQSRNLKPDRRHT
DRDYSLSPSQMNGYSSLQDELLSPASLGCALSSPLRADQYWNEVAVLDAI
PLAATEHDTMLEMSDMQVWSAGLTPSLVTAEDSSLECSKAEDSDATGHEW
KLEGALSEEPRGPELGSLELVEDDTVDSDATNGLIDLLEQEEGQRSEEKL
PGSKRQDDATGAGQDSENEVSLVSGHQRGQARITHSPTVSQVTERSQDRL
QDWDADGSIVSYLQDAAQGSWQEEVTQGPHSFQGTSTMTEGLEPGGSQEY
EKVLVSVSEHTWTEQPEAESSQADRDRRQQGQEEQVQEAKNTFTQVVQGN
EFQNIPGEQVTEEQFTDEQGNIVTKKIIRKVVRQIDLSSADAAQEHEEVT
VEGPLEDPSELEVDIDYFMKHSKDHTSTPNP

A further example nucleotide sequence encoding the ANK1 is the sequence deposited under NCBI Accession No. NM_020476.2.

A further example nucleotide sequence encoding the ANK1 is:

AGAGGCTGCGGTGAGTCCGCCAGCCCCAGCTGCTCCTCCTCAAGCCCCCA
AGGCCCTTCGGCGGGCCCCTGCTGAAAGACCGGCATGCCCTATTCTGTGG
GCTTCCGCGAAGCCGATGCTGCTACCAGCTTTCTGAGAGCAGCAAGATCA
GGTAACTTGGACAAAGCTTTGGATCACCTGCGGAATGGGGTAGATATTAA
CACCTGTAACCAGAATGGGTTGAATGGCTTGCATCTGGCTTCTAAGGAAG
GCCATGTGAAAATGGTGGTTGAACTTCTGCACAAAGAAATCATTCTAGAA
ACGACAACCAAGAAGGGGAACACGGCCCTGCACATCGCTGCTCTAGCCGG
GCAGGATGAGGTGGTCCGGGAGCTTGTCAACTATGGAGCCAACGTCAACG
CCCAGTCACAGAAAGGTTTTACACCCCTGTACATGGCAGCACAAGAGAAC
CACTTGGAAGTGGTTAAGTTTTTACTGGAAAATGGAGCTAACCAGAATGT
AGCCACAGAAGACGGCTTCACGCCTCTGGCGGTAGCCCTGCAGCAGGGCC
ATGAGAACGTCGTCGCGCACCTCATCAACTACGGCACCAAGGGGAAGGTG
CGCCTCCCGGCCCTGCACATCGCGGCCCGCAACGACGACACGCGCACGGC
TGCGGTGCTGCTGCAGAACGACCCCAACCCGGACGTGCTTTCCAAGACGG
GATTCACGCCCCTGCACATTGCGGCTCACTACGAGAACCTCAACGTGGCC
CAGTTGCTCCTCAACAGAGGAGCCAGCGTCAATTTCACACCACAGAACGG
CATCACGCCACTGCACATCGCCTCCCGCAGGGGCAACGTGATCATGGTGC
GGCTGCTGCTGGATCGGGGAGCCCAGATAGAAACCAAGACCAAGGACGAA
TTGACACCTCTCCACTGTGCAGCTCGAAATGGGCACGTGCGAATCTCAGA
GATCCTGCTGGACCACGGGGCACCAATCCAAGCCAAAACCAAGAACGGCC
TGTCCCCAATTCACATGGCGGCTCAGGGAGACCACCTCGACTGTGTCCGG
CTCCTGTTGCAATACGACGCAGAGATAGACGACATCACCCTGGACCACCT
GACCCCACTCCACGTGGCTGCCCACTGTGGACACCACAGGGTGGCTAAGG
TCCTTCTGGATAAAGGGGCCAAACCCAACTCCAGAGCCCTGAATGGCTTT
ACCCCCTTACACATCGCCTGCAAAAAGAACCACGTCCGTGTCATGGAGCT
GCTGCTGAAGACGGGAGCCTCGATCGACGCGGTCACCGAGTCTGGCCTGA
CACCTCTCCACGTGGCCTCCTTCATGGGGCACCTTCCCATCGTGAAGAAC
CTCCTGCAGCGGGGGGCGTCGCCCAACGTCTCCAACGTGAAAGTGGAGAC
CCCGCTACACATGGCAGCCAGAGCCGGGCACACGGAAGTGGCCAAATATT
TACTCCAGAACAAAGCCAAAGTCAATGCCAAGGCCAAGGATGACCAGACC
CCACTTCACTGTGCAGCTCGCATCGGCCACACAAACATGGTGAAGCTCCT
GCTGGAAAATAACGCCAACCCCAACCTGGCCACCACCGCCGGGCACACCC
CCCTGCACATTGCAGCCCGTGAGGGCCATGTGGAAACAGTCCTGGCCCTT
CTGGAAAAGGAAGCATCCCAGGCCTGCATGACCAAGAAAGGATTTACCCC
TCTGCACGTGGCGGCCAAGTACGGGAAGGTGCGGGTGGCAGAGCTGCTGC
TGGAGCGGGACGCACACCCGAATGCTGCCGGAAAAAATGGCCTGACCCCC
CTGCACGTGGCCGTCCATCACAACAACCTGGACATCGTCAAGCTGCTGCT
TCCCCGGGGCGGCTCCCCGCACAGCCCTGCCTGGAATGGCTACACCCCTT
TGCACATCGCTGCCAAGCAGAACCAGGTGGAGGTGGCCCGTAGTCTGCTG
CAGTATGGGGGCTCAGCAAACGCCGAGTCGGTGCAAGGTGTGACGCCCCT
TCACCTGGCCGCCCAGGAGGGCCACGCAGAGATGGTGGCTCTGCTGCTCT
CGAAACAAGCCAATGGCAACCTGGGGAACAAGAGCGGACTCACTCCCCTC
CATCTGGTAGCACAAGAAGGCCACGTTCCAGTGGCAGATGTGCTGATCAA
ACACGGCGTCATGGTGGACGCCACCACCCGGATGGGCTACACTCCCCTCC
ATGTGGCCAGTCACTATGGAAACATCAAGCTGGTGAAGTTTCTGCTGCAG
CACCAGGCAGATGTCAATGCCAAGACCAAGCTAGGATACAGCCCCCTGCA
CCAGGCAGCCCAGCAGGGACACACAGACATCGTGACTCTGCTTCTGAAAA
ACGGTGCTTCCCCAAACGAGGTCAGCTCGGATGGAACCACACCTCTGGCC
ATAGCCAAGCGCTTGGGCTACATTTCTGTCACCGACGTGCTCAAGGTCGT
CACGGATGAAACCAGTTTCGTGTTAGTCAGTGATAAGCATCGAATGAGTT
TCCCTGAGACAGTTGATGAGATCCTGGATGTCTCGGAAGATGAAGGGGAA
GAACTCATCAGCTTCAAGGCTGAGAGGCGGGATTCCAGGGATGTTGATGA
AGAGAAGGAGCTGCTGGATTTTGTGCCGAAGCTAGACCAAGTGGTGGAAT
CTCCAGCCATCCCCAGGATTCCCTGTGCCATGCCTGAGACAGTGGTGATC
AGGTCAGAAGAGCAGGAGCAGGCATCTAAAGAGTATGATGAGGACTCCCT
CATCCCCAGCAGCCCGGCCACCGAGACCTCAGACAACATCAGCCCGGTGG
CCAGCCCGGTGCATACAGGGTTTCTGGTGAGCTTCATGGTTGACGCCCGG
GGTGGTTCCATGAGAGGAAGTCGCCACAACGGCCTGCGAGTGGTGATCCC
GCCACGGACGTGCGCAGCGCCCACCCGCATCACCTGCCGCCTGGTCAAGC
CCCAGAAGCTCAGCACGCCGCCCCCACTGGCCGAGGAGGAGGGCCTGGCC
AGCAGGATCATAGCACTGGGGCCCACGGGGGCACAGTTCCTGAGCCCTGT
AATCGTGGAGATCCCGCACTTTGCCTCCCATGGCCGTGGAGACCGCGAGC
TCGTGGTTCTGAGGAGCGAAAACGGCTCCGTGTGGAAGGAGCACAGGAGC
CGCTATGGAGAGAGCTACCTGGATCAGATCCTCAACGGGATGGACGAAGA
GCTGGGGAGCCTGGAGGAGCTAGAGAAGAAGAGGGTGTGCCGAATCATCA
CCACCGACTTCCCGCTGTACTTCGTGATCATGTCACGGCTCTGCCAGGAC
TACGACACCATCGGTCCCGAAGGGGGCTCCCTGAAGAGCAAGCTGGTGCC
CCTGGTACAGGCAACGTTCCCGGAGAATGCCGTCACCAAGAGAGTGAAGC
TGGCTCTGCAGGCCCAGCCTGTCCCGGATGAGCTTGTCACTAAGCTCCTG
GGCAACCAGGCCACATTCAGCCCCATTGTCACCGTGGAGCCCCGGCGCCG
GAAGTTCCACCGCCCCATTGGGCTTCGGATCCCACTACCTCCTTCCTGGA
CCGACAACCCGAGGGACAGCGGGGAGGGAGACACCACCAGCCTGCGCCTG
CTTTGCAGCGTCATTGGAGGAACAGACCAAGCCCAGTGGGAAGACATAAC
AGGAACCACCAAACTTGTATATGCCAACGAGTGCGCCAACTTCACCACCA
ATGTCTCTGCCAGGTTTTGGCTGTCGGACTGTCCTCGGACTGCTGAGGCT
GTGAACTTTGCCACCCTGCTGTACAAAGAGCTCACTGCAGTGCCCTACAT
GGCCAAATTCGTCATCTTTGCCAAGATGAATGACCCCCGAGAGGGGCGCC
TGCGCTGCTACTGCATGACAGATGATAAAGTGGACAAGACCCTGGAGCAG
CATGAGAACTTCGTGGAGGTGGCCCGGAGCAGGGACATAGAGGTGTTGGA
AGGAATGTCCCTGTTTGCAGAACTCTCTGGGAACCTGGTGCCTGTGAAGA
AAGCTGCCCAGCAGCGGAGCTTCCACTTCCAGTCATTTCGGGAGAACCGT
CTGGCCATGCCTGTAAAGGTGAGGGACAGCAGTCGAGAGCCGGGAGGGTC
CCTGTCGTTTCTGCGCAAGGCGATGAAGTACGAGGACACCCAGCACATTC
TCTGCCACCTGAACATCACCATGCCCCCCTGCGCCAAGGGAAGTGGAGCC
GAAGATAGGAGAAGGACCCCGACGCCCCTGGCCCTGCGATACAGCATTCT
CAGTGAGTCCACACCAGGTTCTCTCAGTGGGACAGAGCAGGCAGAGATGA
AGATGGCTGTTATCTCAGAGCACCTCGGTCTCAGCTGGGCAGAGTTGGCC
CGGGAGCTGCAGTTCAGTGTGGAAGACATCAACAGGATCCGAGTGGAAAA
TCCCAACTCCCTGTTGGAGCAGAGTGTGGCCTTGCTGAACCTCTGGGTCA
TCCGTGAAGGCCAAAACGCAAACATGGAGAATCTGTACACAGCCCTGCAG
AGCATTGACCGTGGCGAGATCGTGAACATGCTGGAGGGTTCCGGCCGACA
GAGCCGCAACTTGAAGCCAGACAGGCGGCACACCGACCGCGACTACTCGC
TGTCACCCTCCCAGATGAATGGTTACTCCTCACTGCAGGACGAGCTGCTG
TCCCCTGCCTCCCTGGGCTGTGCACTTTCCTCTCCGCTACGTGCAGACCA
GTACTGGAATGAGGTGGCCGTCCTAGACGCCATCCCCTTGGCGGCCACGG
AGCATGACACCATGCTGGAGATGTCTGACATGCAGGTGTGGTCTGCGGGC
CTCACGCCTTCTCTGGTCACTGCTGAGGACTCCTCTCTGGAGTGTAGCAA
GGCTGAGGACTCTGATGCCACAGGTCACGAGTGGAAGTTGGAGGGGGCAC
TCTCAGAGGAACCGCGGGGCCCCGAGTTGGGCTCTCTGGAACTTGTGGAG
GACGACACAGTGGATTCAGATGCCACAAATGGCCTTATCGATTTGCTTGA
ACAGGAGGAAGGTCAGAGGTCAGAAGAGAAGCTGCCAGGTTCTAAGAGGC
AGGATGACGCGACAGGTGCAGGGCAGGACTCAGAGAATGAAGTGTCTCTT
GTTTCAGGCCATCAGAGGGGGCAAGCCCGAATCACACATTCCCCCACCGT
GAGTCAGGTGACGGAGAGGAGTCAGGACAGACTGCAGGACTGGGATGCAG
ACGGCTCGATTGTCTCATACCTGCAAGATGCTGCACAAGGTTCCTGGCAA
GAGGAGGTCACGCAAGGTCCACACTCATTCCAGGGAACAAGTACCATGAC
TGAAGGGCTAGAGCCCGGTGGATCTCAGGAGTACGAGAAGGTCCTGGTGT
CTGTAAGTGAGCACACGTGGACAGAACAGCCCGAGGCTGAGAGCTCCCAG
GCCGACAGGGACCGGAGGCAGCAAGGCCAAGAAGAGCAGGTGCAGGAGGC
CAAGAACACCTTCACCCAAGTGGTGCAGGGGAATGAGTTTCAGAATATTC
CAGGGGAGCAGGTGACAGAGGAGCAATTCACGGATGAGCAGGGCAACATT
GTCACCAAGAAGATCATTCGCAAGGTGGTTCGACAGATAGACTTGTCCAG
CGCCGATGCCGCCCAGGAGCACGAGGAGGTGACTGTAGAGGGGCCCCTGG
AGGATCCCAGTGAGCTGGAGGTCGATATTGATTACTTTATGAAACACTCC
AAGGATCACACCTCGACACCCAACCCCTGAACCCCACACACTCTGCCATG
CACACAGGAGGAGAGCTGGACCTGAGGGCCACCGCAGCGGTGCACACATT
CCTCTGGGCTGACGGCATGACCTCTGTAAGGGACTCCTGCTAGTCCCCTC
TTGGCATGAATGACTGACTGTAGACGCATGACCTCCAGGCTTCAATCCTG
CCTCTTGCAATGACAGCTGATCTGTCGGAACCAGGACACAAAAGCAGCAA
GAAGCGGGGAGAGAGAGGGATAGAAAACAAGCGCAGGAGAGCCTGCGAAC
GCAAAAGTGAATGAGGGCTTTTTGTGGCTGGGGATGGGTTTTGGTTTTGG
GGTTTTTTTTTTAAATTGTTTTGACTTCGTACAGGGTACTTTTTCCCAAC
CTCATCTGTCAGAAATCCATGTGGGCTTCCTGGAAAGAAAAAAAAAAAAA
AGAAAACTAGGCATGAAATCAGTTTAACACCTTAATCTTAAGCCATGTCC
TCATCTGCCCACCCTCCCCAACCCACATACCTCCATTCCACTTGTGACAC
CCCCTCGACTCCCTGGTGACGCTCCTCCCAGATCGCTCTACATGACATTC
AGGACACACACACACACACACACACACCACTCGCCTCCACTGAATCTACA
CACAGATTTTACTGTGACTTCTGAAGCTGTATAGACTCTGCTGTGGATAA
ATTGGAATTTTTTATGTTGTCTCTCTCTCTGCCAATTTCAATACGAATCA
TCTTCCAATGGAAAATCATTACCTTGAAGAGTGCATTCGGGGTTCCTTGT
GTTAGGGACTTAAGAATCTGAGGCGAGGACCCCCAGGCTTACCTGTAGGA
CTCAAAGGGAGCCAGGACCCCATCTGAAAGGGTCTCCTCTCTCAGTTGGG
GGACAGGCCGGTGGCTGACCCAGGATTGCACCAGCATGTCCATAGAGAAG
AGGTTTTCTATGTCTTCAAGCACTATATCATAGTCCGTGTTCAAAGTGTA
AACTGTACAGTAATCAGCCTTGTGTATATGAAAAACAATAAATACTATGC
AAACCAATAGAAACATTTAGCAGTACGTACAGAGCCTGACAGCGCAGCTC
CTGAGGACTTCTGCGGCTGCAGGAGAAGGAGCTGTGGCCTGTCTTTCAGT
GAAAGAGGAAGGAAAGGAGTAGGGCTAGTGAATGTATCTGCCAGGTCTTA
GAAGTCAGGACCACCCAGGGTCTCCCAGAGTGAACTTGGTGCTCTAAGTC
CAGGCAGCCTGCAGGCTGGGGACCCTGGGTGCAGGTGCCAGGCTCGAGGC
TGTTCCCTGTCCTGCCTCAGTGCCTCCCCTCTGCCCAGCCCCTGTGGCCT
CTTGGAGAGGCGAGCCAGGGGGCTCCCCGGACCGCATGGCCGGTGAGAGT
AGAGTCGGAGGCGGAGAGCCTGGACGCCCAGAGGCCGGGGCCAGGCTCTG
GGTCAAGGCAGGAAGGAGACAGACAGCTGGCAGTTCCTCGCCTCGGTGCC
ATCAGGGAGGAGCCTTAAGTTCCACTAGAGCCTCCGGCCCAGGGAGGCCA
GAATCAGCACAATCCCGGCCCCGCCCTCGCGGCGGGCGCCCCCAGGGCCA
GGAGAGAGAGACAGGGCTGGCTCCTCTCGCCCCTGGAGCCGCGGTGTGGA
CGGCCCGGCCCCGTCCGCCCCGCCGCTCTGCGGGAGTGTCCTCGGGACAC
ACTTAAACCTTCCAAGTAGCACAGAAGCCCCCTCCACGTCAGAGGCCCCC
TGGCCGCGGGGCCTGTGAGCGGAGAGGGGCCCTGCTTCCCACACAAATCC
GCCCCGGTGCACCCCCGGCCAGGCAGGCCCCGGGGCGGCCTAGGCGCCTT
GTCGGCCGGGGGTGGGGCTGCTTTTGCTTCCTTTAGAGGGGCGGGCAGGG
AGAGGAAGCTCTAGCTCCGGGCCTGAGTTTCCCCGAGGCTGCGTCGGAGG
AGCTGCAGGGCGAGCCCGGACGGCTGCCGTCTCCGCCAGAGGCCACCCCG
GCCCGGCCTCCAGGGCGCAGAGGCGCCCCCCTCCATCCTCCCCCATCCCG
GGGGCCACGGCAGGGGGGCCGCACAGCGCGGCTGGGACCGATTCGGTGAC
GGCCCAAGGAAGCTGGGCTCAGGGCTCGCCTCTCCCGCCTCGATCCTGCA
CCTTCCTCTCCCAGGTCGCTGCTGCAGCCAACCCAGTAGCCCCCTTAGCG
CCCCCGCGGACCCCGCAGCTCCAGACTCCGCTTTGGCACCTCTGCTCCGG
CTGGCATGGCTGCACGCTCCGGCTGCTAGTAGGAGCCGTTAGGGGAGGAG
TTTGGGGTCTCCACACGATGCCTAGAGAATGCTGCAGTCTGCACATTAGA
CGCTTTTTAGAAGTTTTGAAATTACCTTGATTTTTTTAATTGTTATGAAA
ATGGATCTTTTCTTGACTCTCCCACATGCTCTGTTATGGGAGAGAATCCC
CTACCCTACTCTGATGTATAGACCATTCTCCCTTCACCAGCCGAACCAAT
GTCAAAATTAATAAAGAAATGGACTAATGGCAAAAAAAAA

A further example amino acid sequence of the ANK1 is the sequence deposited under NCBI Accession No. NP_065211.2.

A further example amino acid sequence of the ANK1 is:

(SEQ ID NO: 5)
MWTFVTQLLVTLVLLSFFLVSCQNVMHIVRGSLCFVLKHIHQELDKELGE
SEGLSDDEETISTRVVRRRVFLKGNEFQNIPGEQVTEEQFTDEQGNIVTK
KIIRKVVRQIDLSSADAAQEHEEVELRGSGLQPDLIEGRK GAQIVKRAS
LKRGKQ

A further example nucleotide sequence encoding the ANK1 is the sequence deposited under NCBI Accession No. NM_020478.4.

A further example nucleotide sequence encoding the ANK1 is:

(SEQ ID NO: 6)
GTCACCCGGGCCACTCAGGGCAGAGCCTGGCTGCTGGGCAGAAGGGCACT
TGGGCCGGGCTGCTTCAGCCGAGAGGGCCATTCGGTGTCCTGCCCACAGA
GAGAGAAACAGTGAGAGACCAAGAGAGCAAACCAGAGAGATAGGGAGGGG
GAATGCAAGGGGTCCCTGGGGGACTCAGGAGTCCCAGTGGAGAATGAGAG
GCAGGCCCCCCGTGGTGGCCGGGGCCCTGGCTGCCAGAGGACCGGGCTGG
TGAGGAGGCGAGGATGTGGACTTTCGTCACCCAGCTGTTGGTCACGCTGG
TGCTGCTGAGCTTCTTCCTGGTCAGCTGTCAGAACGTGATGCACATTGTC
AGGGGGTCCCTGTGCTTTGTGCTAAAGCACATCCACCAGGAGCTGGACAA
GGAGCTGGGGGAGAGCGAGGGCCTCAGTGACGACGAGGAGACCATCTCCA
CCAGGGTGGTCCGGCGGCGGGTCTTCCTGAAGGGGAATGAGTTTCAGAAT
ATTCCAGGGGAGCAGGTGACAGAGGAGCAATTCACGGATGAGCAGGGCAA
CATTGTCACCAAGAAGATCATTCGCAAGGTGGTTCGACAGATAGACTTGT
CCAGCGCCGATGCCGCCCAGGAGCACGAGGAGGTGGAGCTGAGAGGGAGT
GGCCTACAGCCGGACCTGATAGAGGGCAGGAAGGGGGCGCAGATAGTGAA
GCGGGCCAGCCTGAAAAGGGGGAAACAGTGACCCCGAGCCGCTCTCCTTG
GAGTAGCCTCTCGGGAGGATCACACCTCGACACCCAACCCCTGAACCCCA
CACACTCTGCCATGCACACAGGAGGAGAGCTGGACCTGAGGGCCACCGCA
GCGGTGCACACATTCCTCTGGGCTGACGGCATGACCTCTGTAAGGGACTC
CTGCTAGTCCCCTCTTGGCATGAATGACTGACTGTAGACGCATGACCTCC
AGGCTTCAATCCTGCCTCTTGCAATGACAGCTGATCTGTCGGAACCAGGA
CACAAAAGCAGCAAGAAGCGGGGAGAGAGAGGGATAGAAAACAAGCGCAG
GAGAGCCTGCGAACGCAAAAGTGAATGAGGGCTTTTTGTGGCTGGGGATG
GGTTTTGGTTTTGGGGTTTTTTTTTTAAATTGTTTTGACTTCGTACAGGG
TACTTTTTCCCAACCTCATCTGTCAGAAATCCATGTGGGCTTCCTGGAAA
GAAAAAAAAAAAAAAGAAAACTAGGCATGAAATCAGTTTAACACCTTAAT
CTTAAGCCATGTCCTCATCTGCCCACCCTCCCCAACCCACATACCTCCAT
TCCACTTGTGACACCCCCTCGACTCCCTGGTGACGCTCCTCCCAGATCGC
TCTACATGACATTCAGGACACACACACACACACACACACACCACTCGCCT
CCACTGAATCTACACACAGATTTTACTGTGACTTCTGAAGCTGTATAGAC
TCTGCTGTGGATAAATTGGAATTTTTTATGTTGTCTCTCTCTCTGCCAAT
TTCAATACGAATCATCTTCCAATGGAAAATCATTACCTTGAAGAGTGCAT
TCGGGGTTCCTTGTGTTAGGGACTTAAGAATCTGAGGCGAGGACCCCCAG
GCTTACCTGTAGGACTCAAAGGGAGCCAGGACCCCATCTGAAAGGGTCTC
CTCTCTCAGTTGGGGGACAGGCCGGTGGCTGACCCAGGATTGCACCAGCA
TGTCCATAGAGAAGAGGTTTTCTATGTCTTCAAGCACTATATCATAGTCC
GTGTTCAAAGTGTAAACTGTACAGTAATCAGCCTTGTGTATATGAAAAAC
AATAAATACTATGCAAACCAATAGAAACATTTAGCAGTACGTACAGAGCC
TGACAGCGCAGCTCCTGAGGACTTCTGCGGCTGCAGGAGAAGGAGCTGTG
GCCTGTCTTTCAGTGAAAGAGGAAGGAAAGGAGTAGGGCTAGTGAATGTA
TCTGCCAGGTCTTAGAAGTCAGGACCACCCAGGGTCTCCCAGAGTGAACT
TGGTGCTCTAAGTCCAGGCAGCCTGCAGGCTGGGGACCCTGGGTGCAGGT
GCCAGGCTCGAGGCTGTTCCCTGTCCTGCCTCAGTGCCTCCCCTCTGCCC
AGCCCCTGTGGCCTCTTGGAGAGGCGAGCCAGGGGGCTCCCCGGACCGCA
TGGCCGGTGAGAGTAGAGTCGGAGGCGGAGAGCCTGGACGCCCAGAGGCC
GGGGCCAGGCTCTGGGTCAAGGCAGGAAGGAGACAGACAGCTGGCAGTTC
CTCGCCTCGGTGCCATCAGGGAGGAGCCTTAAGTTCCACTAGAGCCTCCG
GCCCAGGGAGGCCAGAATCAGCACAATCCCGGCCCCGCCCTCGCGGCGGG
CGCCCCCAGGGCCAGGAGAGAGAGACAGGGCTGGCTCCTCTCGCCCCTGG
AGCCGCGGTGTGGACGGCCCGGCCCCGTCCGCCCCGCCGCTCTGCGGGAG
TGTCCTCGGGACACACTTAAACCTTCCAAGTAGCACAGAAGCCCCCTCCA
CGTCAGAGGCCCCCTGGCCGCGGGGCCTGTGAGCGGAGAGGGGCCCTGCT
TCCCACACAAATCCGCCCCGGTGCACCCCCGGCCAGGCAGGCCCCGGGGC
GGCCTAGGCGCCTTGTCGGCCGGGGGTGGGGCTGCTTTTGCTTCCTTTAG
AGGGGCGGGCAGGGAGAGGAAGCTCTAGCTCCGGGCCTGAGTTTCCCCGA
GGCTGCGTCGGAGGAGCTGCAGGGCGAGCCCGGACGGCTGCCGTCTCCGC
CAGAGGCCACCCCGGCCCGGCCTCCAGGGCGCAGAGGCGCCCCCCTCCAT
CCTCCCCCATCCCGGGGGCCACGGCAGGGGGGCCGCACAGCGCGGCTGGG
ACCGATTCGGTGACGGCCCAAGGAAGCTGGGCTCAGGGCTCGCCTCTCCC
GCCTCGATCCTGCACCTTCCTCTCCCAGGTCGCTGCTGCAGCCAACCCAG
TAGCCCCCTTAGCGCCCCCGCGGACCCCGCAGCTCCAGACTCCGCTTTGG
CACCTCTGCTCCGGCTGGCATGGCTGCACGCTCCGGCTGCTAGTAGGAGC
CGTTAGGGGAGGAGTTTGGGGTCTCCACACGATGCCTAGAGAATGCTGCA
GTCTGCACATTAGACGCTTTTTAGAAGTTTTGAAATTACCTTGATTTTTT
TAATTGTTATGAAAATGGATCTTTTCTTGACTCTCCCACATGCTCTGTTA
TGGGAGAGAATCCCCTACCCTACTCTGATGTATAGACCATTCTCCCTTCA
CCAGCCGAACCAATGTCAAAATTAATAAAGAAATGGACTA ATGGCAAAA
AAAAA

In one embodiment, the ANK1 comprises an amino acid sequence that has at least 70, 80%, 85%, 90%, 95%, 96%, 97%, 98%, 99% or 100% identity to SEQ ID NO: 1 or 3 or 5, preferably wherein the amino acid sequence substantially retains the natural function of the protein represented by SEQ ID NO: 1 or 3 or 5.

In one embodiment, the ANK1-encoding nucleotide sequence comprises a nucleotide sequence that has at least 70%, 80%, 85%, 90%, 95%, 96%, 97%, 98%, 99% or 100% identity to SEQ ID NO: 2 or 4 or 6, preferably wherein the protein encoded by the nucleotide sequence substantially retains the natural function of the protein represented by SEQ ID NO: 1 or 3 or 5.

In one embodiment, the ANK1-encoding nucleotide sequence comprises a nucleotide sequence that encodes an amino acid sequence that has at least 70%, 80%, 85%, 90%, 95%, 96%, 97%, 98%, 99% or 100% identity to SEQ ID NO: 1 or 3 or 5, preferably wherein the amino acid sequence substantially retains the natural function of the protein represented by SEQ ID NO:1 or 3 or 5.

### Weight Loss and Weight Maintenance

The term “weight loss” as used herein may refer to a reduction in parameters such as weight (e.g. in kilograms), body mass index (kg/m2), waist-hip ratio (e.g. in centimetres), fat mass (e.g. in kilograms), hip circumference (e.g. in centimetres) or waist circumference (e.g. in centimetres).

Weight loss may be calculated by subtracting the value of one or more of the aforementioned parameters at the end of an intervention (e.g. a diet and/or exercise regimen) from the value of the parameter at the onset of the intervention.

The degree of weight loss may be expressed as a percent change of one of the aforementioned weight phenotype parameters (e.g. a percent change in a subject's body weight (e.g. in kilograms) or body mass index (kg/m²)). For example, a subject may lose at least 10% of their initial body weight, at least 8% of their initial body weight, or at least 5% of their initial body weight. By way of example only, a subject may lose between 5 and 10% of their initial body weight.

In one embodiment, a degree of weight loss of at least 10% of initial body weight results in a considerable decrease in the risk of obesity-related co-morbidities.

The term “weight maintenance” as used herein may refer to the maintenance in parameters such as weight (e.g. in kilograms), body mass index (kg/m²), waist-hip ratio (e.g. in centimetres) fat mass (e.g. in kilograms), hip circumference (e.g. in centimetres) or waist circumference (e.g. in centimetres). Weight maintenance may refer to, for example, maintaining weight lost following an intervention (e.g. a diet and/or exercise regimen).

The degree of weight maintenance may be calculated by determining the change in one or more of the afore-mentioned parameters over a period of time. The period of time may be, for example, at least 5, 10, 15, 20, 25, 30, 35, 40, 45 or 50 weeks.

Weight maintenance supported by the agents of the invention may result in, for example, a change (e.g. gain) of less than 10%, 9%, 8%, 7%, 6%, 5%, 4%, 3%, 2% or 1% in one or more of the afore-mentioned parameters over a period of time.

The degree of weight maintenance may be expressed as the weight regained during a period following attainment of weight loss, for example as a percentage of the weight lost during attainment of weight loss.

Weight maintenance supported by the agents of the invention may result through suppression of a subject's appetite following administration of the agent. The subject may therefore have a reduced appetite compared to the appetite in the absence of the agent of the invention.

Weight maintenance supported by the agents of the invention may result through control of a subject's appetite following administration of the agent. The subject may therefore maintain control over their appetite and therefore maintain their weight, for example following a period of weight loss intervention.

In particular, the agents of the invention may support weight maintenance through appetite suppression or control during and/or following a period of weight loss intervention (e.g. a diet or exercise regime).

In one aspect, the invention provides the non-therapeutic use of an agent of the invention to (i) reduce fat accumulation and/or (ii) preserving or increasing fat free mass in a subject.

### Obesity

The term “overweight” as used herein is defined for an adult human as having a body mass index (BMI) between 25 and 30.

The term “body mass index” as used herein means the ratio of weight in kg divided by the height in metres, squared.

The term “obesity” as used herein refers to a condition in which the natural energy reserve, stored in the fatty tissue of animals, in particular humans and other mammals, is increased to a point where it is associated with certain health conditions or increased mortality. The term “obese” as used herein is defined for an adult human as having a BMI greater than 30.

The term “normal weight” as used herein is defined for an adult human as having a BMI of 18.5 to 25, whereas the term “underweight” as used herein may be defined as a BMI of less than 18.5.

Obesity is a chronic metabolic disorder that has reached epidemic proportions in many areas of the world and is the major risk factor for serious co-morbidities such as type 2 diabetes mellitus, cardiovascular disease, dyslipidaemia and certain types of cancer (World Health Organ. Tech. Rep. Ser. (2000) 894: i-xii, 1-253).

The term “obesity-related disorder” as used herein refers to any condition which an obese individual is at an increased risk of developing. Obesity-related disorders include diabetes (e.g. type 2 diabetes), stroke, high cholesterol, cardiovascular disease, insulin resistance, coronary heart disease, metabolic syndrome, hypertension and fatty liver.

### Methods of Screening

The invention provides agents that are capable of decreasing the activity of ANK1, and additionally provides methods for identifying such agents.

The agents of the invention may be identified by methods that provide either qualitative or quantitative results. Furthermore, such methods may be used to characterise as well as identify agents of the invention.

The candidate agents may be any agents of potential interest, for example peptides, polypeptides (e.g. antibodies), nucleic acids or small molecules. Preferably, the candidate agents are compounds or mixtures of potential therapeutic interest. Preferably, the candidate agents are of low toxicity for mammals, in particular humans. In some embodiments, the candidate agents may comprise nutritional agents and/or food ingredients, including naturally-occurring compounds or mixtures of compounds such as plant or animal extracts.

The candidate agents may form part of a library of agents, for example a library produced by combinatorial chemistry or a phage display library. In one embodiment, the candidate agents form part of a library of plant bioactive molecules.

### ANK1 Activity

The ability of a candidate agent to reduce the activity of a protein, for example an enzyme, may be expressed in terms of an IC50 value. The IC50 is the concentration of an agent that is required to give rise to a 50% reduction in the activity of the protein (e.g. a 50% reduction in enzymatic activity). The calculation of IC50 values is well known in the art.

Preferably, the agents of the invention have an IC50 value for inhibition of ANK1 of less than 100 μM, more preferably less than 10 μM, for example less than 1 μM, less than 100 nM or less than 10 nM.

Techniques for measuring ANK1 activity may be applied to ANK1 that has been isolated from a cell. The ANK1 may have been expressed using recombinant techniques. Preferably, the ANK1 has been purified.

### ANK1 Binding

The invention also provides methods of identifying agents which are capable of binding to ANK1 and, alternatively or additionally, characterising such binding. For example, the method may allow measurement of absolute or relative binding affinity, and/or enthalpy and entropy of binding. Binding affinity may be expressed in terms of the equilibrium dissociation (K_{d}) or association (Kₐ) constant.

A number of assay techniques are known in the art for identifying binding between a candidate agent and a protein. The assay technique employed is preferably one which is amenable to automation and/or high throughput screening of candidate agents. The assay may be performed on a disposable solid support such as a microtitre plate, microbead, resin or similar.

For example, target ANK1 may be immobilised on a solid support, for example a microbead, resin, microtitre plate or array. Candidate agents may then be contacted with the immobilised target protein. Optionally, a wash procedure may be applied to remove weakly or non-specifically binding agents. Any agents binding to the target protein may then be detected and identified. To facilitate the detection of bound agents, the candidate agents may be labelled with a readily detectable marker. The marker may comprise, for example, a radio label, an enzyme label, an antibody label, a fluorescent label, a particulate (e.g. latex or gold) label or similar.

Alternatively, the above procedure may be reversed and the candidate agents may be immobilised and the target ANK1 may be contacted with said immobilised agents. Optionally, a wash procedure may be applied to remove weakly or non-specifically bound target protein. Any agents to which ANK1 binds may then be detected and identified. To facilitate the detection of binding, the ANK1 may be labelled with a readily detectable marker as described above.

In addition to the assays described above, other suitable assay techniques are known in the art. Examples of such techniques include radioassays, fluorescence assays, ELISA, fluorescence polarisation, fluorescence anisotropy, isothermal titration calorimetry (ITC), surface plasmon resonance (SPR) and the like. These assays may be applied to identify agents which bind to ANK1. Indeed, platforms for the automation of many of these techniques are widely known in the art to facilitate high-throughput screening.

More than one assay technique may be used to provide a detailed understanding of a candidate agent's binding to ANK1. For example, assays which provide qualitative binding information may be used as a first step in the method, followed by further assays using different techniques to provide quantitative binding data and/or data on the effect on activity of the target protein.

The assay techniques described above may be adapted to perform competition binding studies. For example, these techniques are equally suitable to analyse the binding of a protein to substrate or cofactor in the presence of a candidate agent. It will therefore be possible to use the above techniques to screen and identify agents that modulate the binding between a protein and its substrate or cofactor, thus having an effect on the protein's activity.

Preferably, the agents of the invention will bind with high affinity. For example, the agents of the invention will bind to ANK1 with a K_{d} of less than 100 μM, more preferably less than 10 μM, for example less than 1 μM, less than 100 nM or less than 10 nM.

Binding affinity may be measured using standard techniques known in the art, e.g. surface plasmon resonance, ELISA and so on (for instance as described above), and may be quantified in terms of either dissociation (K_{d}) or association (Kₐ) constants.

Bioinformatics-based approaches, such as in silico structure-guided screening, may also be used to identify agents of the invention.

### ANK1 Levels

The invention provides agents for decreasing ANK1 levels. Levels of ANK1 may be equated with levels of expression of the protein in a cell or organism. Protein levels may be analysed directly or indirectly, for example by analysis of levels of mRNA encoding the protein.

Methods for analysing the expression of ANK1 may be employed in the invention to screen the effect of a candidate agent on the protein's levels.

A number of techniques are known in the art for determining the expression level of a protein. These techniques may be applied to test the effect of candidate agents on the expression level of ANK1. The technique employed is preferably one which is amenable to automation and/or high throughput screening of candidate agents.

For example, screens may be carried out using cells harbouring polynucleotides encoding ANK1 operably linked to a reporter moiety. The reporter moiety may be operably linked to endogenous ANK1-encoding genes. Alternatively, exogenous copies of ANK1 operably linked to a reporter moiety may be inserted into a cell. In this embodiment, the cell may be engineered to be deficient for natural ANK1 expression. Suitable reporter moieties include fluorescent labels, for example fluorescent proteins such as green, yellow, cherry, cyan or orange fluorescent proteins.

The term “operably linked” as used herein means the components described are in a relationship permitting them to function in their intended manner.

Such cells may be contacted with candidate agents and the level of expression of ANK1 may be monitored by analysing the level of reporter moiety expression in the cell. Fluorescent reporter moieties may be analysed by a number of techniques known in the art, for example flow cytometry, fluorescence activated cell sorting (FACS) and fluorescence microscopy. Expression levels of ANK1 may be compared before and after contact with the candidate agent. Alternatively, expression levels of ANK1 may be compared between cells contacted with a candidate agent and control cells.

Other methods may be used for analysing the expression of proteins, for example ANK1. Protein expression may be analysed directly. For example, expression may be quantitatively analysed using methods such as SDS-PAGE analysis with visualisation by Coomassie or silver staining. Alternatively, expression may be quantitatively analysed using Western blotting or enzyme-linked immunosorbent assays (ELISA) with antibody probes which bind the protein product. ANK1 labelled with reporter moieties, as described above, may also be used in these methods. Alternatively, protein expression may be analysed indirectly, for example by studying the amount of mRNA corresponding to the protein that is transcribed in a cell. This can be achieved using methods such as quantitative reverse transcription PCR and Northern blotting.

Similar techniques may also be used for the analysis of leptin protein expression.

### Agents

The invention provides agents that are capable of decreasing the activity of ANK1, and additionally provides methods for identifying such agents.

The agents of the invention may be, for example, peptides, polypeptides (e.g. antibodies), nucleic acids (e.g. siRNAs, shRNAs, miRNAs and antisense RNAs) or small molecules. Preferably, the agents are of low toxicity for mammals, in particular humans. In some embodiments, the agents may comprise nutritional agents and/or food ingredients, including naturally-occurring compounds or mixtures of compounds such as plant or animal extracts.

Example agents that decrease or otherwise affect the activity of ANK1 include the agents recited in Table 1.

TABLE 1
Agents that decrease or otherwise affect the activity of ANK1. (Davis AP, et al. The
Comparative Toxicogenomics Database: update 2017. Nucleic Acids Res. 2016 Sep. 19)

| Chemical Name | Chemical ID | CAS RN | Interaction Actions | Reference |
|---|---|---|---|---|
| Acetaminophen | D000082 | 103-90-2 | affects expression | Toxicol Lett. 2014 Oct. |
| | | | | 1; 230(1): 48-56. |
| Amiodarone | D000638 | 1951-25-3 | affects expression | Toxicol Lett. 2014 Oct. |
| | | | | 1; 230(1): 48-56. |
| AZM551248 | C547126 | | decreases expression | Toxicol Sci. 2012 |
| | | | | May; 127(1): 236-45. |
| Benzo(a)pyrene | D001564 | 50-32-8 | decreases expression | Toxicol Sci. 2012 |
| | | | | April; 126(2): 391-404. |
| Carbamazepine | D002220 | 298-46-4 | affects expression | Toxicol Sci. 2015 |
| | | | | August; 146(2): 311-20. |
| Chlorpromazine | D002746 | 50-53-3 | affects expression | Toxicol Lett. 2014 Oct. |
| | | | | 1; 230(1): 48-56. |
| Cyclosporine | D016572 | 59865-13-3 | affects expression | Toxicol Lett. 2014 Oct. |
| | | | | 1; 230(1): 48-56. |
| decabromobiphenyl | C010902 | 1163-19-5 | decreases expression | Arch Toxicol. 2013 |
| ether | | | | August; 87(8): 1557-67. |
| Dexamethasone | D003907 | 50-02-2 | decreases expression | Endocrinology. 2010 |
| | | | | March; 151(3): 1050-9. |
| Dieldrin | D004026 | 60-57-1 | decreases expression | Neurotoxicology. 2013 |
| | | | | January; 34: 105-17. |
| Ethinyl Estradiol | D004997 | 57-63-6 | decreases expression | Mol Pharmacol. 2008 |
| | | | | January; 73(1): 82-93. |
| Gentamicins | D005839 | | decreases expression | Toxicol Appl Pharmacol. 2012 |
| | | | | Jan. 1; 258(1): 124-33. |
| oxaliplatin | C030110 | | decreases expression | Front Genet. 2015; 6: 14. |
| Propylthiouracil | D011441 | 51-52-5 | decreases expression | Toxicol Lett. 2014 Aug. |
| | | | | 4; 228(3): 225-34. |
| Tamoxifen | D013629 | 10540-29-1 | affects expression | BMC Genomics. 2007; 8: 151. |
| Tetracycline | D013752 | 60-54-8 | affects expression | Toxicol Lett. 2014 Oct. |
| | | | | 1; 230(1): 48-56. |
| Thioacetamide | D013853 | 62-55-5 | affects expression | Toxicol Lett. 2014 Oct. |
| | | | | 1; 230(1): 48-56. |
| titanium dioxide | C009495 | 13463-67-7 | decreases expression | Toxicol Appl Pharmacol. 2013 |
| | | | | Jun. 15; 269(3): 250-62. |
| Topotecan | D019772 | 123948-87-8 | decreases expression | Front Genet. 2015; 6: 14. |
| Valproic Acid | D014635 | 99-66-1 | affects expression | Toxicol Sci. 2015 |
| | | | | August; 146(2): 311-20. |

In one embodiment, the agent is generally regarded as safe (GRAS).

In one embodiment, the agent has a bioavailability of not less than 50%, when administered orally.

The agent for use according to the invention may be selected from Table 1.

In one embodiment, the agent is Acetaminophen. In one embodiment, the agent is Amiodarone. In one embodiment, the agent is AZM551248. In one embodiment, the agent is Benzo(a)pyrene. In one embodiment, the agent is Carbamazepine. In one embodiment, the agent is Chlorpromazine. In one embodiment, the agent is Cyclosporine. In one embodiment, the agent is decabromobiphenyl ether. In one embodiment, the agent is Dexamethasone. In one embodiment, the agent is Dieldrin. In one embodiment, the agent is Ethinyl Estradiol. In one embodiment, the agent is Gentamicins. In one embodiment, the agent is oxaliplatin. In one embodiment, the agent is Propylthiouracil. In one embodiment, the agent is Tamoxifen. In one embodiment, the agent is Tetracycline. In one embodiment, the agent is Thioacetamide. In one embodiment, the agent is titanium dioxide. In one embodiment, the agent is Topotecan. In one embodiment, the agent is Valproic Acid.

In one embodiment, the agent for use according to the invention is an agent selected from Table 1 and which decreases activity or expression of ANK1.

The agents for use according to the invention may be, for example, present as salts or esters, in particular pharmaceutically acceptable salts or esters.

siRNAs, shRNAs, miRNAs and Antisense DNAs/RNAs

Expression of ANK1 may be modulated using post-transcriptional gene silencing (PTGS). Post-transcriptional gene silencing mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11: 429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA>30 bp has been found to activate the interferon response leading to shut-down of protein synthesis and non-specific mRNA degradation (Stark et al. (1998) Ann. Rev. Biochem. 67: 227-64). However, this response can be bypassed by using 21 nt siRNA duplexes (Elbashir et al. (2001) EMBO J. 20: 6877-88; Hutvagner et al. (2001) Science 293: 834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) non-coding RNAs that can effectively reduce the translation of target mRNAs by binding to their 3′ untranslated region (UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein, and methods for the delivery of these agents to a cell of interest are well known in the art. Furthermore, methods for specifically modulating (e.g. reducing) expression of a protein in a certain cell type within an organism, for example through the use of tissue-specific promoters are well known in the art.

### Antibodies

The term “antibody” as used herein refers to complete antibodies or antibody fragments capable of binding to a selected target, and includes Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

Methods for the production of antibodies are known by the skilled person. Alternatively, antibodies may be derived from commercial sources.

If polyclonal antibodies are desired, a selected mammal (e.g. mouse, rabbit, goat or horse) may be immunised. Serum from the immunised animal may be collected and treated according to known procedures. If the serum contains polyclonal antibodies to other antigens, the polyclonal antibodies may be purified by immunoaffinity chromatography. Techniques for producing and processing polyclonal antisera are known in the art.

Monoclonal antibodies directed against antigens (e.g. proteins) used in the invention can also be readily produced by the skilled person. The general methodology for making monoclonal antibodies by hybridomas is well known. Immortal antibody-producing cell lines can be created by cell fusion and also by other techniques such as direct transformation of B-lymphocytes with oncogenic DNA or transfection with Epstein-Barr virus. Panels of monoclonal antibodies produced against antigens can be screened for various properties, for example for isotype and epitope affinity.

An alternative technique involves screening phage display libraries where, for example, the phage express scFv fragments on the surface of their coat with a large variety of complementarity determining regions (CDRs). This technique is well known in the art.

Antibodies, both monoclonal and polyclonal, which are directed against antigens, are particularly useful in diagnosis, and those which are neutralising are useful in passive immunotherapy. Monoclonal antibodies in particular may be used to raise anti-idiotype antibodies. Anti-idiotype antibodies are immunoglobulins which carry an “internal image” of the antigen of the infectious agent against which protection is desired.

Techniques for raising anti-idiotype antibodies are known in the art. These anti-idiotype antibodies may also be useful for treatment, as well as for an elucidation of the immunogenic regions of antigens.

Introduction of Polypeptides and Polynucleotides into Cells

An agent for use in the invention may be, for example, a polypeptide or a polynucleotide. Polynucleotides and polypeptides may also need to be introduced into cells as part of the methods or screening assays of the invention.

Where the invention makes use of a polypeptide, the polypeptides may be administered directly to a cell (e.g. the polypeptide itself may be administered), or the polypeptides may be administered by introducing polynucleotides encoding the polypeptide into cells under conditions that allow for expression of the polypeptide in a cell of interest. Polynucleotides may be introduced into cells using vectors.

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred to a target cell. The vector may serve the purpose of maintaining the heterologous nucleic acid (e.g. DNA or RNA) within the cell, facilitating the replication of the vector comprising a segment of nucleic acid or facilitating the expression of the protein encoded by a segment of nucleic acid. Vectors may be non-viral or viral. Examples of vectors used in recombinant nucleic acid techniques include, but are not limited to, plasmids, chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid or virus vectors and may include a promoter for the expression of a polynucleotide and optionally a regulator of the promoter.

Vectors comprising polynucleotides used in the invention may be introduced into cells using a variety of techniques known in the art, such as transduction and transfection. Several techniques suitable for this purpose are known in the art, for example infection with recombinant viral vectors, such as retroviral, lentiviral, adenoviral, adeno-associated viral, baculoviral and herpes simplex viral vectors; direct injection of nucleic acids and biolistic transformation. Non-viral delivery systems include, but are not limited to, DNA transfection methods. Transfection includes a process using a non-viral vector to deliver a gene to a target cell.

Transfer of the polypeptide or polynucleotide may be performed by any of the methods known in the art which may physically or chemically permeabilise the cell membrane. Cell-penetrating peptides may also be used to transfer a polypeptide into a cell.

In addition, the invention may employ gene targeting protocols, for example the delivery of DNA-modifying agents.

The vector may be an expression vector. Expression vectors as described herein comprise regions of nucleic acid containing sequences capable of being transcribed. Thus, sequences encoding mRNA, tRNA and rRNA are included within this definition.

Expression vectors preferably comprise a polynucleotide for use in the invention operably linked to a control sequence that is capable of providing for the expression of the coding sequence by the host cell. A regulatory sequence “operably linked” to a coding sequence is ligated in such a way that expression of the coding sequence is achieved under conditions compatible with the control sequence. The control sequence may be modified, for example by the addition of further transcriptional regulatory elements to make the level of transcription directed by the control sequence more responsive to transcriptional modulators.

### Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides, such as DNA polynucleotides, may be produced recombinantly, synthetically or by any means available to the skilled person. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA, for example mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

### Proteins

The term “protein” as used herein includes single chain polypeptide molecules as well as multiple-polypeptide complexes where individual constituent polypeptides are linked by covalent or non-covalent means. The terms “polypeptide” and “peptide” as used herein refer to a polymer in which the monomers are amino acids and are joined together through peptide or disulfide bonds.

### Variants, Derivatives, Analogues, Homologues and Fragments

In addition to the specific proteins and nucleotides mentioned herein, the invention also encompasses variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3, to 10 or 20 substitutions, provided that the modified sequence retains the required activity or ability.

Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells, as well as for a variety of other organisms.

### Method of Treatment

All references herein to treatment include curative, palliative and prophylactic treatment. The treatment of mammals, particularly humans, is preferred. Both human and veterinary treatments are within the scope of the invention.

### Administration

Although the agents for use in the invention can be administered alone, they will generally be administered in admixture with a pharmaceutical carrier, excipient or diluent, particularly for human therapy.

In some embodiments, the agent is a nutritional agent, food additive or food ingredient, and may thus be formulated in a suitable food composition. Thus, the agent may be administered, for example, in the form of a food product, drink, food supplement, nutraceutical, nutritional formula or pet food product.

### Dosage

The skilled person can readily determine an appropriate dose of an agent of the invention to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1: Link Between ANK1 Genetic Variants and Weight Loss

This study relates to a genetic association performed on Diogenes weight loss intervention data and the Optifast900 Canadian study.

The Diogenes study is a pan-European, randomised and controlled dietary intervention study investigating the effects of dietary protein and glycaemic index on weight loss and weight maintenance in obese and overweight families in eight European centres (Larsen et al. (2009) Obesity Rev. 11: 76-91). In brief, the Diogenes study included overweight/obese subjects that followed an 8-week low-caloric diet (LCD). The LCD provided 800 kcal per day with the use of a meal-replacement product (Modifast, Nutrition et Sante France).

The Optifast900 Canadian study consisted of patients enrolled in the Weight Management Clinic who had completed 6 to 12 weeks meal-replacement regimen consisting of a product uniquely available in Canada, Optifast900 (Nestle Health Science, Switzerland).

This is the first study that has tested the association between common variants genotyped on an Illumina chip and protein expression change during intervention focusing on proteins associated to body mass index (BMI) change.

Genotype data were generated using HumanCoreExome-12 v1.1 with 264,909 tag SNP marker and 244,593 exome-focused markers (www.illumina.com). They were processed with the Illumina TM platform following Infinium® HD Assay Ultra, Manual according to manufacturer's instructions. Genotypes were called with the GenomeStudio Software (Illumina). Quality control procedure followed recommendations from the GenABEL package (Aulchenko, Y. S., Ripke, S., Isaacs, A. & van Duijn, C. M. Bioinforma. Oxf. Engl. 23, 1294-1296 (2007) and excluded SNPs with call rate <95%, violating Hardy-Weinberg equilibrium (FDR<20%), low minor allele frequency <1%. Subjects were excluded if they had low call rate (<95%), abnormally high autosomal heterozygosity (FDR<1%), an XXY karyotype, or gender inconsistencies between genotype data and clinical records. For subjects with high identity-by-state (IBS>95%), only the one having the highest call rate was kept. Principal component analyses (PCA) were performed independently on each cohort to discard subjects that were outliers in term of genetic structure. Subjects from both cohorts were all of European ancestry and the two cohorts had similar genetic structure. Upon all genetic QCs, 1166 Ottawa and 798 DiOGenes subjects were kept for subsequent analyses. Genotype imputation was then performed using SHAPEIT (Delaneau, O., Marchini, J. & Zagury, J.-F. Nat Meth 9, 179-181 (2012)) and IMPUTE2 (Howie, B. N., Donnelly, P. & Marchini, J. PLoS Genet. 5, e1000529 (2009)) based on the European reference panel from the 1000 Genome project (Abecasis, G. R. et al. Nature 491, 56-65 (2012)) (March 2012 release, phase 1 version 3). Imputation post-filtering removed SNPs with reference allele frequency less than 1% and INFO score <0.8. Single-SNP analyses were performed using BOLT-LMM (Loh, P.-R. et al. Nat. Genet. 47, 284-290 (2015)), a Bayesian linear mixed effect model to adjust for population structure and cryptic relatedness between individuals. The rate of weight loss was adjusted for sex, age and starting BMI. Results from the two cohorts, were subsequently meta-analyzed using Genome-Wide Association Meta Analysis (GWAMA) software (Msgi, R. & Morris, A. BMC Bioinformatics 11, 288 (2010)) with random-effect modeling and a double genomic-control (GC) correction (Devlin, B. & Roeder, K. Biometrics 55, 997-1004 (1999)) (GC correction at study-level and also at meta-analysis level).

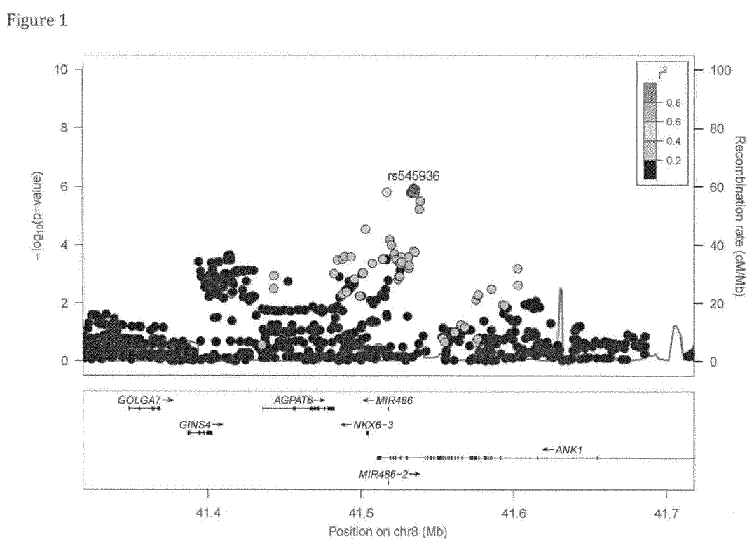

SNPs were found to be associated with weight loss upon caloric restriction (FIG. 1). For example, the rs545936 variant (with global MAF=19% and located at position 41534485 on chromosome 8 (GRCh37 coordinate)) emerged as the top associated SNP.

### Example 2: Prioritization of ANK1 Genetic Variants

Prioritization of GWA signals was performed using a Bayesian framework to model the joint likelihood of association p-values with large-scale epigenomic annotations. Such risk variance inference was performed using the RiVIERA-beta framework (Li, Y. & Kellis, M. Nucleic Acids Res. 44, e144 (2016)) and with 450 epigenomic annotations (including histone marks, DNase I hypersensitivity, transcription factor binding, and localization within exons). The goal of this framework is to infer for each input SNP the posterior probability of disease given its association p-value and overlap in functional annotations. Epigenomic annotations were retrieved from Pickrell et al. (Am. J. Hum. Genet. 94, 559-573 (2014)).

The benefit from such modelling is to identify which variant(s) may be the most plausible(s) in term of causal/regulatory effect, even if those variants were not necessarily the top associated ones (i.e. having the most extreme p-values). And indeed, from such modelling, the rs6981587 SNP (with global MAF=34% and located at position 41516915 on chromosome 8 (GRCh37 coordinate)) emerged as the most likely causal variant. Specifically, each added copy of the C allele from rs6981587 was associated with better weight loss. As expected, other variants, in linkage disequilibrium with rs6981587 also ranked with good probability of being causal SNPs.

### Example 3: In Vivo Function of ANK1

Fly strains. Fly stocks were maintained on standard diet with agar, sugar and yeast and were raised in 25° C. incubator at a 12/12 dark and night cycle. Actin-Ga/4 was from Bloomington and w1118 and UAS-Ank^{IR} (GD 25945) were from the VDRC.

Triglyceride assay. 10 (4-7 days old) male flies were weighted and homogenised in 200 μl dH₂O on ice, then sonicated for 10 s using a probe sonicator on ice. After sonication, 800 μl ice-cold dH₂O was added and mixed thoroughly. 50 μl of the mixture was used to determine the triglycerides using Roche triglycerides kits (11730711216) under manufacturer's instructions. Body weight was measured by analytic balance. Triglycerides were normalized to body weight.

Fly body length measurements: 4-7 days old male flies were C02 knockdown and then taken photo using Zeiss microscope. The fly body length was measured in ImageJ Fiji with an internal standard length marker.

qPCR for RNAi knockdown efficiency. RNA was extracted from 4-7 days old male as standard protocol. All extracted RNA met the quality for qPCR (A260/A280>2.0, A260/A230>2.0). 1 μg of mRNA was reversed into cDNA using SuperScript® III First-Strand Synthesis System (Invitrogen). All primers were prescreened for efficiency and specificity. The RT-PCR was performed using Sensimix™ probe kit (Bioline). The program is following: 95° C. 10 min, 40 cycles of 95° C., 15 s; 55° C., 15 s; 72° C., 15 s. The reactions were run on LightCycle® 480 (Roche).

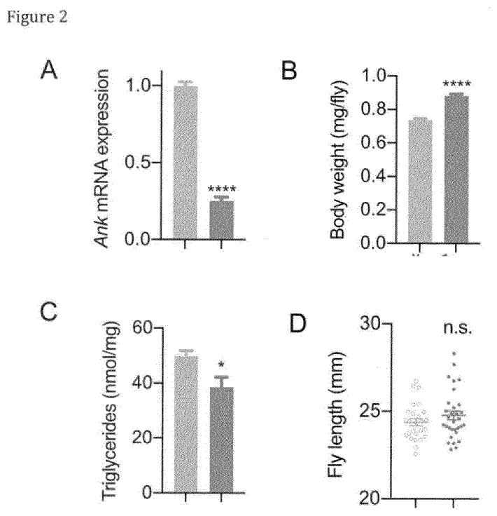

To investigate ANK1 gene function in vivo we used transgenic RNAi in the fruit fly Drosophila melanogaster. Protein alignment between ANK1 and the fly homologue Ank is 61%, which is considered a high orthology score. Using whole body (Actin-Ga/4) driver, animals were viable with no overt developmental phenotype and we observed ˜75% knock down of Ank mRNA (Actin-Ga/4>UAS-Ank^{IR}) compared to controls (Actin-Ga/4/+) (FIG. 2A). Importantly, Ank knockdown animals exhibited marked (20%) increase in body weight (FIG. 2B), while triglycerides levels were significantly (23%) lower in these animals (FIG. 2C) with no change in body size (FIG. 2D). Thus, targeting Ank in vivo promotes a lean phenotype in the fly.

All publications mentioned in the above specification are herein incorporated by reference. Various modifications and variations of the disclosed agents, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for reducing fat accumulation and/or preserving or increasing fat free mass in a subject comprising the step of administering an agent capable of decreasing the activity of ANK1 to a subject in need of same.

2. The method according to claim 1, wherein the agent is capable of decreasing the activity of ANK1 for improving dislipidemia.

3. The method according to claim 1, wherein the agent is capable of reducing the risk of cardiovascular disease (CVD).

4. The method according to claim 1, wherein the agent is administered to a subject during or after a weight loss intervention, preferably during a weight loss intervention.

5. The method according to claim 1, wherein the agent decreases the level of ANK1 in a subject.

6. The method according to claim 1, wherein the agent is selected from the agents listed in Table 1.

7. The method according to claim 1, wherein the agent is selected from the group consisting of an siRNA, shRNA, miRNA, antisense RNA, polynucleotide, polypeptide and a small molecule.

8. A method of identifying an agent capable of (i) reducing fat accumulation and/or (ii) preserving or increasing fat free mass in a subject comprising the steps:
(a) contacting a preparation comprising a ANK1 polypeptide or polynucleotide with a candidate agent; and
(b) detecting whether the candidate agent affects the activity of the ANK1 polypeptide or polynucleotide.

9. A method of identifying an agent that decreases the activity of ANK1 comprising the steps:
(a) contacting a preparation comprising a ANK1 polypeptide or polynucleotide with a candidate agent; and
(b) detecting whether the candidate agent affects the activity of the ANK1 polypeptide or polynucleotide.

10. The method of claim 8, wherein the preparation comprising the ANK1 polypeptide or polynucleotide comprises a cell comprising the ANK1 polypeptide or polynucleotide.

11. The method of claim 10, wherein the cell is an adipocyte.

12. The method of claim 8, wherein the method is for identifying an agent that decreases the expression of ANK1.

13. The method of claim 8, wherein the candidate agent is a natural product.

14. The method of claim 9, wherein the preparation comprising the ANK1 polypeptide or polynucleotide comprises a cell comprising the ANK1 polypeptide or polynucleotide.

15. The method of claim 10, wherein the cell is an adipocyte.

16. The method of claim 9, wherein the method is for identifying an agent that decreases the expression of ANK1.

17. The method of claim 9, wherein the candidate agent is a natural product.
